(19) United States
(12) Patent Application Publication
(10) Pub. No.: US 2022/0016064 A1
(43) Pub. Date: Jan. 20, 2022

(54) VERY LONG CHAIN FATTY ACID COMPOSITIONS

(71) Applicant: EPAX NORWAY AS, Ålesund (NO)

(72) Inventors: Harald SVENSEN, Ålesund (NO); Björn KRISTINSSON, Kopavogur (IS)

(21) Appl. No.: 17/311,600

(22) PCT Filed: Dec. 5, 2019
(86) PCT No.: PCT/NO2019/050268

§ 371 Date: Jun. 7, 2021

(30) Foreign Application Priority Data
Dec. 6, 2018 (NO) 20181574

(51) Int. Cl. A61K 31/201; A61K 31/202; A61K 35/60; C11B 3/00; A23L 33/12

(52) U.S. Cl. CPC A61K 31/201; A61K 31/202; A23V 2002/00; C11B 3/003; A23L 33/12; A61K 35/60

(57) ABSTRACT

The invention relates to compositions comprising fatty acid mixtures comprising very long chain unsaturated fatty acids. The fatty acids of the fatty acid mixture are isolated from natural oils. Particularly, the fatty acid mixtures comprise an enriched amount of both very long chain monounsaturated fatty acids (VLCMUFAs) and very long chain polyunsaturated fatty acids (VLCPUFAs). In one embodiment of the invention, the amount of cholesterol in the fatty acid mixture is minimised and a method for production is provided.

### FIELD OF THE INVENTION

The present invention relates to fatty acid mixtures comprising very long chain unsaturated fatty acids. Particularly, the invention provides compositions comprising such fatty acid mixtures wherein the amount of both very long chain monounsaturated fatty acids and very long chain polyunsaturated fatty acids have been enriched.

### BACKGROUND OF THE INVENTION

Among the long-chain fatty acids, the long chain polyunsaturated fatty acids (LCPUFAs), and especially long-chain omega-3 fatty acids (LCn3), the fatty acids of chain length C20-C22, have received the most interest in literature. The acronyms EPA (for eicosapentaenoic acid) and DHA (for docosahexaenoic acid) have become household names in describing valuable omega-3-acids from fish oil and other sources. Products rich in alpha-linoleic acid (ALA) from plant sources are also available in the market. More recently, the long-chain monounsaturated fatty acids (LCMUFAs) with chain length C20-C22 have come into the focus of scientific interest. See, for example, Breivik and Vojnovic, Long chain monounsaturated fatty acid composition and method for production thereof, U.S. Pat. No. 9,409,851B2.

In this regard, it is noted that lipids are described by the formula X:YnZ wherein X is the number of carbon atoms in their alkyl chain, and Y is the number of double bonds in such chain; and where “nZ” is the number of carbon atoms from the methyl end group to the first double bond. In nature, the double bonds are all in the cis-form. In polyunsaturated fatty acids each double bond is separated from the next by one methylene (—CH2) group. Using this nomenclature, EPA is 20:5n3; DHA is 22:6n3 and ALA is C18:3n3, while C20:1n9 and C22:1n11 represent the most abundant LCMUFAs in North Atlantic fish oils.

Further, natural sources of omega-3 fatty acids, such as fish oil, also comprise fatty acids of shorter and longer length than C20-C22. As is employed herein, the term very long chain fatty acids (or VLCFAs) is intended to mean fatty acids (or FAs) having a chain length of more than 22 carbon atoms; the term very long chain polyunsaturated fatty acids (or VLCPUFAs) is intended to mean polyunsaturated fatty acids (or PUFAs) having a chain length of more than 22 carbon atoms; the term very long chain monounsaturated fatty acids (or VLCMUFAs) is intended to mean monounsaturated fatty acids (or MUFAs) having a chain length of more than 22 carbon atoms; while the term VLCn3 is intended to refer to polyunsaturated omega-3 fatty acids having a chain length of more than 22 carbon atoms, it being understood that VLCn3 represents a sub-group of VLCPUFA. The term very long chain saturated fatty acids (VLCSFAs) is intended to mean saturated fatty acids (or SFAs) having a chain length of more than 22 carbon atoms. The term very long chain unsaturated fatty acids (or VLCUSFAs) is intended to mean unsaturated fatty acids having a chain length of more than 22 carbon atoms, i.e. unsaturated fatty acids having a chain length of 24 carbon atoms or more, and encompass both VLCMUFAs and VLCPUFAs.

In order to produce marine omega-3-concentrates rich in EPA and DHA, conventional industrial processes are designed to concentrate the polyunsaturated C20-C22 fraction, by removing both short-chain fatty acids as well as molecules larger than the C22 fatty acids. Examples of such processes are molecular/short path distillation, urea fractionation, extraction and chromatographic procedures, all of which can be utilized to concentrate the C20-22 fraction of marine fatty acids and similar materials derived from other sources. A review of these procedures is provided in Breivik H (2007) Concentrates. In: Breivik H (ed) Long-Chain Omega-3 Specialty Oils. The Oily Press, PJ Barnes & Associates, Bridgwater, UK, pp 111-140. Some procedures for the concentration of C20-C22 MUFAs are given in U.S. Pat. No. 9,409,851B2.

Polyunsaturated fatty acids are very liable to oxidation. In order to comply with pharmacopoeia and voluntary standards imposing upper limits for oligomeric/polymeric oxidation products, it is common to remove components with chain length above that of DHA, for example by distillation, extraction and similar procedures. Further, such higher molecular weight components of marine oils are typically associated with undesirable unsaponifiable constituents of such oil including cholesterol as well as with organic pollutants such as brominated diphenyl ethers.

However, biologically active PUFAS, including omega-3 acids, are not limited to the C20 and C22 chain lengths of EPA and DHA. According to the American Oil Chemist's' Society's Lipid Library, VLCPUFAs of both the omega-3 and omega-6 families occur in the retina, brain and sperm. In 2014 the American Oil Chemist's' Society's Lipid Library was up-dated with a review on the metabolism of VLCPUFAs in mammals. This review gives information that VLCPUFAs are isolated within the mammalian body to retinal tissue, testes, brain, and spermatozoa. Further, this review provides very useful information on valuable physiological roles of VLCPUFAs, including their importance for optimal functioning of the eyes and cerebral tissues as well as for male fertility. On the other hand, the review states that, unlike LCPUFAs, VLCPUFAs cannot be obtained from dietary sources, and thus must be synthesised in situ from shorter chain fatty acid precursors.

As a consequence of this belief, much work has focused upon the production of VLCPUFAs using recombinant techniques. For example, Anderson et al (US 2009/0203787A1) disclose a recombinant process for producing C28-C38 VLCPUFAs using the ELOVL4 gene. Also, Katavic et al. (WO2008/061334) disclose that seed oils with elevated levels of the VLCMUFA C24:1n9, and to a small extent also C26:1n9, may be recovered from transgenic seeds. In a more recent book chapter, Bennett and Anderson state that the importance of VLCPUFAs in treating diseases in the retina would be solidified if VLCPUFAs could be reconstituted in the deficient retinas. “However, VLCPUFAs cannot be chemically synthesised in large enough quantities to allow feeding studies in mice”, (Bennett LD and Anderson RE (2016) Current Progress in Deciphering Importance of VLC-PUFA in the retina. In: C. Bowes Rickman et al. (eds.) Retinal Degenerative Diseases, Advances in Experimental Medicine and Biology 854, Springer, Switzerland). This concern demonstrates why there have been no studies which involve adding VLCPUFAs to animal feed, and even less so any clinical studies on humans. To the best of our knowledge, Breivik and Svensen in WO2016/182452 were the first to disclose a method for producing compositions of very long chain polyunsaturated fatty acids (VLCPUFAs), specifically very long chain omega-3 fatty acids (VLCn3s), as well as compositions comprising high concentrations of such VLCn3s, from natural oils. Breivik and Svensen further disclose that there is only a small amount of the VLCn3s in natural oils like fish oils and explain why these and other very long chain fatty acids are substantially removed during production of traditional marine omega-3 concentrates, where the aim is to up-concentrate omega-3-fatty acids with chain length C20-C22.

Omega-3 fatty acids, and particularly the LCPUFAs EPA and DHA, are known to have a broad range of beneficial health effects and are hence known for different uses. As stated above, more recently the marine C20-C22 LCMUFAs have also become known to have beneficial health effects. Further, there are also indications that VLCMUFAs may have beneficial health effects. For example, mice deficient in enzymes for elongation to very long chain fatty acids (i.e. VLCMUFA and VLCPUFA) display scaly and wrinkled skin and a severely damaged epidermal barrier functioning, and die within a few hours after birth (Vasireddy et al. (2007) Human Molecular Genetics, 2007, Vol. 16, No. 5 471-482 doi:10.1093/hmg/dd1480), indicating a function and the need for the VLCUSFAs.

It has been indicated that very long chain unsaturated fatty acids do have beneficial health effects. There is hence a need for the provision of compositions of the VLCFAs in order to meet the body's need for these fatty acids. However, there is a very limited amount of VLCUSFAs found in few known biological sources, and the known compositions comprising such VLCUSFAs are either synthesised from shorter fatty acid precursors, they are from processes using recombinant techniques, or they are from transgenic plants.

### BRIEF SUMMARY OF THE INVENTION

The invention provides compositions comprising fatty acid mixtures of both very long chain monounsaturated fatty acids and very long chain polyunsaturated fatty acids. The very long chain unsaturated fatty acids derive from natural oils and the amount of these fatty acids has been enriched.

In one aspect the composition comprises a fatty acid mixture, wherein the fatty acid mixture comprises both very long chain monounsaturated fatty acids and very long chain polyunsaturated fatty acids, and further wherein the amount of cholesterol in the fatty acid mixture is minimised.

The invention further provides a method for production of a composition comprising a fatty acid mixture comprising both VLCPUFAs and VLCMUFAs, wherein the fatty acid mixture is prepared from an oil material, the method comprising the steps of:
i) converting free cholesterol present in the oil material to cholesterol esters; and
ii) separating the cholesterol esters of step i) from very long chain fatty acid esters present in the oil material of step i).

### DETAILED DESCRIPTION OF THE INVENTION

The compositions of the invention comprise a fatty acid mixture of an enriched amount of very long chain unsaturated fatty acids. Particularly, the fatty acid mixtures comprise an enriched amount of both very long chain monounsaturated fatty acids (VLCMUFAs) and very long chain polyunsaturated fatty acids (VLCPUFAs). In one embodiment, the amount of cholesterol in the fatty acid mixture is particularly low.

The fatty acid mixture of the composition comprises mainly fatty acids, and preferably at least 90.0%, such as at least 95.0% by weight of the fatty acid mixture is different fatty acids. The prepared composition is hence preferably an oil composition, an may also be called a fatty acid composition or an enriched composition, wherein this composition comprises an enriched amount of both VLCMUFAs and VLCPUFAs.

In a first embodiment, the composition comprises a fatty acid mixture of at least 0.5%, such as at least 1.0%, such as at least 2.0% by weight of VLCMUFAs. More preferably, the fatty acid mixture comprises at least 4% by weight of VLCMUFAs, such as more than 5%, more than 8%, more than 15%, more than 20%, more than 30%, more than 40%, more than 50% or even more than 60% by weight of very long chain monounsaturated fatty acids. In one embodiment, the fatty acid mixture comprises VLCMUFAs in an amount of 4.0-50.0%, such as 8.0-50.0% by weight of the fatty acid mixture.

The VLCMUFAs, and any other VLCFAs, of the fatty acid mixtures have a chain length of more than 22 carbon atoms. Hence, the VLFAs are herein defined to have a chain length of 24 carbon atoms or more. In one embodiment, the fatty acid mixtures of the compositions comprise at least one VLCMUFA with a chain length of 24 carbons or longer.

Preferably, the compositions comprise a mixture of different such VLCUSFAs, both monounsaturated and polyunsaturated. In this regards, the VLCUSFAs may have chain lengths of 24, 26, 28, 30, 32, 34, 36, 38, 40 or 42 carbons. Preferably, the VLCUSFAs of the composition are a mixture of fatty acids with chain lengths of 24, 26, 28, 30 and 32 carbon atoms. In one embodiment, the fatty acid mixture comprises at least 1%, such at least 3%, such as at least 6%, such as at least 10% of VLCMUFAs with a chain length of more than 24 carbon atoms, i.e. with 26 or more carbon atoms.

VLCMUFAs that may be present in the compositions are selected from any one of, including but not limited to, the following group of fatty acids: C24:1 (tetraconsenoic acid (nervonic acid)), C26:1 (hexacosenoic acid), C28:1 (octacosenoic acid), C30:1 and C32:1.

In one embodiment, the amount of C28:1 fatty acid is low, and the amount of this specific VLCMUFA is less than 4.0%, less than 3.0%, less than 2.0%, such as less than 1.0%, and preferably less than 0.5% by weight of the fatty acid mixtures.

For the VLCMUFAs, the applicant has been able to isolate them from the raw oil, and increase the amount compared to the content of the same VLCMUFAs in the raw oil used. In one embodiment, the fatty acid mixtures of the compositions comprise any of these VLCMUFAs in the listed amounts:

C24:1: 4.0-50.0%, such as 7.0-40.0%, 8.0-20.0%, such 13.0-20.0%, such as about 40%;

C26:1: 1.0-20.0%, such as 6.0-20.0, such 10.0-18.0, more particularly 11.0-15.0%, such as about 13%.

The compositions of the invention are enriched compositions, wherein unsaturated fatty acids, and particularly VLCUSFAs, have been isolated, and the concentration has been increased compared to the content of these in the raw oil used. Hence, the enriched compositions are composed of desired fatty acids which have been selected, sorted and concentrated from the raw material.

The fatty acid mixtures of the compositions comprise, in addition to the very long chain monounsaturated fatty acids, further very long chain polyunsaturated acids. These polyunsaturated fatty acids may include 2, 3, 4, 5, 6, 7 or 8 double bonds.

In one embodiment, the fatty acid mixture comprises VLCPUFAs in at least 0.5%, such as at least 1%, at least 2%, at least 5%, at least 8%, or at least 10% by weight of the fatty acid mixture. In one embodiment, the fatty acid mixture comprises VLCPUFAs in an amount of 5.0%-40.0%, such as 7.0-15.0 by weight of the fatty acid mixture. The fatty acid mixture may comprise at least 5.0%, 8.0%, 10%, 20%, 30%, 40%, 50%, or even at least 60% VLCPUFAs by weight of the fatty acid mixture.

By way of example, the fatty acid mixtures of the compositions may in addition to the VLCMUFAs comprise either of the following groups of VLCPUFAs in the listed amounts, or any combinations of these:

C24 VLCPUFAS: At least 1.0%: Such as 1.0-20.0%, such as 2.0-12.0%; or

C26 VLCPUFAs: 0.5-30.0%; such as 1.0-12.0%; or

C28 VLCPUFAs: 1.0-70.0%, such as 2.0-30.0%, such as at least 5%, or

C32 VLCPUFAs: 0.0-5.0%.

C32-C40 VLCPUFAs: 0.0-5.0%.

For VLCPUFAs, the applicant has been able to isolate these from the raw oil and to increase the amount of these compared to the content of the same VLCPUFAs in the raw oil used. In one embodiment, the fatty acid mixtures of the compositions comprise any of the following

VLCPUFAs in the listed amounts, or any combinations of these:

C24:5 n3: 1.0-10.0%, such as about 5%;

C26:4 n3: 1.0-6.0%, such as about 4%;

C26:5 n3: 1.0-7.0%, such as about 5%;

C26:6 n3: at least 5%, such as 5.0-20.0%, such as about 10%;

C28:4 n3: 1.0-5.0%, such as about 3%;

C28:5 n3: 0.5-3.0%, such as about 2%;

C28:6 n3: 2.0-10.0%, such as about 6%;

C28:7 n3: 0.5-2.0%, such as about 1%;

C28:8 n3: 4.0-60%, such as about 40%;

C30:5, C30:6 and C30:8: 0.5-2.0%; and

C32-C40 PUFAs: 0.0-5.0%.

In one embodiment, the fatty acid mixture comprises at least 4%, such as at least 5%, such as about 4-50% of C28 VLCPUFAs. In a particular embodiment, the fatty acid mixture comprises either of C28:6n3, C28:7n3 and C28:8n3 fatty acids in a total amount of at least 5% by weight of the fatty acid mixture. Hence, fatty acid mixtures comprising at least 5% by weight; at least 8% by weight or at least 10% by weight of C28:6, C28:7 and/or C28:8 very long chain polyunsaturated fatty acids can be produced. At the same time fractions enriched in C28:4n3, C28:5n3 and/or C28:6n3 may also be produced. Similarly, fatty acid mixtures enriched in C24:5n3 and/or C24:6n3 may also be produced, and in one embodiment the fatty acid mixture comprises at least 5% by weight of C24:5n3 fatty acids. Further, in another particular embodiment, the fatty acid mixture comprises at least 5% of C26 VLCPUFAs, such as at least 5% of the C26:6n3 VLCPUFA.

The VLCPUFAs are e.g. omega-3, omega-6 or omega-9 fatty acids, and are preferably omega-3 or omega-6 fatty acids, and most preferably they are omega-3 PUFAs. In particular embodiments, the compositions comprise VLCPUFAs selected from any one of, including but not limited to, the following group of fatty acids: C24:5n3, C26:6n3, C28:6n3, C28:8n3. In one embodiment, the fatty acid mixture comprises at least 10% omega-3 VLCPUFAs by weight of the fatty acid mixture.

Accordingly, the composition of the invention comprises both very long chain mono- and polyunsaturated fatty acids. In one embodiment, the fatty acid mixture of the composition comprises at least 4% VLCMUFAs, such as at least 8% VLCMUFAs and at least 1% VLCPUFAs. In one embodiment, the fatty acid mixtures of the compositions comprise at least 9.0% VLCUSFAs by weight of the fatty acid mixture, such as more than 10%, more than 15%, more than 20%, more than 30%, more 40%, more than 50%, more than 60%, more than 70%, more than 80% or even more than 90% VLCUSFA by weight of the fatty acid mixtures. In one embodiment, the fatty acid mixture comprises at least 1% by weight of very long chain unsaturated fatty acids (VLCUSFAs) with chain length of more than 24 carbon atoms, such as at least 1% by weight of very long chain monounsaturated fatty acids with chain length of more than 24 carbon atoms. The VLCUSFA content is the combined amount of VLCMUFAs and VLCPUFAs. In one embodiment, the weight ratio between the VLCMUFAs and VLCPUFAs in the fatty acid mixture, such as between VLCMUFAs and omega-3 VLCPUFAs, is preferably in the range of 3:1-1:2.

With a high concentration of the VLCUSFAs present, there is naturally a lower concentration of short and long chain fatty acids. However, some of the long chain unsaturated fatty acids may be present in the composition, particularly such fatty acids having beneficial health effects. In one embodiment, in addition to the VLCUSFAs, the fatty acid composition comprises one or more LCPUFA, such as one or more C20-C22 PUFAs. In certain embodiments, the fatty acid mixtures comprise at least 5% LCPUFAs by weight of the fatty acid mixture, such as at least 10%, at least 20%, at least 25%, at least 30%, at least 40%, at least 50%, or at least 60% by weight of at least one LCPUFA, such as one or more C20-C22 long chain PUFAs. In one embodiment, the LCPUFAs comprise at least one of EPA, DHA and omega-3 DPA (all-cis-7,10, 13, 16,19-docosapentaenoic acid, 22:5n3). Further, in other embodiments, the fatty acid mixtures of the compositions comprise at least 5%, at least 8%, or at least 10% by weight of omega-3 DPA (22:5n3). In some embodiments of the present invention, the weight ratio of EPA:DHA of the composition ranges from about 1:15 to about 10:1, from about 1:10 to about 8:1, from about 1:8 to about 6:1, from about 1:5 to about 5:1, from about 1:4 to about 4:1, from about 1:3 to about 3:1, or from about 1:2 to about 2:1. In one embodiment, the fatty acid mixtures of the compositions comprise at least 8% VLCMUFAs, at least 1% VLCPUFAs and at least 5% LCPUFAs, by weight of the fatty acid mixture.

Further, the compositions of the invention may comprise C18 and long chain monounsaturated fatty acids (LCMUFAs), and the fatty acid mixture comprises in one embodiment at least 1% by weight of C18-C22 MUFAs, such as at least 1% by weight of C20-22 MUFAs. In one embodiment, the fatty acid mixture comprises at least 1% by weight of VLCMUFAS and at least 1% by weight of VLCPUFAs derived from natural oils, and where the fatty acid mixture further comprises at least 10% by weight of C20-C22 monounsaturated fatty acids. Such MUFAs of the composition are selected from the group of, including but not limited to, the following group of fatty acids; oleic acid (C18:1 n9), vaccenic acid (C18:1 n7), gondoic acid (C20:1 n9), gadoleic acid (C20:1 n11), erucic acid (C22:1 n9) and cetoleic acid (C22:1 n11). In one embodiment, the amount of erucic acid is below 8.0%, such as below 5%, preferably below 3% and more preferably below 2% by weight of the fatty acid mixtures of the compositions. In another embodiment, the amount of oleic acid is below 5.0%, and more preferably below 2% by weight of the fatty acid mixtures of the compositions.

Further, the acetylenic acids, comprising a triple bond, such as xymenynic acid (018H3002, C18:1) named trans-11-octadecen-9-ynoic acid, is preferably not present in the fatty acid mixture, and the amount of acetylenic acids is less than 0.1% by weight of fatty acid mixture. Further, the fatty acid mixtures enriched with VLCUSFAs preferably comprise a low amount of saturated fatty acids, of all lengths. Although for some applications it may be of some benefit to include very long chain saturated fatty acids (VLCSFAs), such as in a concentration above 2%, the amount should preferably be kept low. In total, the fatty acid mixtures comprise less than 1.0% saturated fatty acids, more preferably less than 0.5% saturated fatty acids. Particularly, the amount of C16:0 (palmitic acid), C18:0 (stearic), and C20:0 (arachidic acid) is low, and preferably the content of these, in total, is less than 1.0%. Particularly, the amount of stearic acid is low, and is preferably below 1.0%, and more preferably below 0.5%. Further, the amount of very long chain saturated fatty acids (VLCSFA) is low, and the amount of the fatty acids C24:0, C26:0, C28:0 and C30:0 is preferably in total below 2.0%, more preferably below 1.0% and most preferably below 0.5% by weight of the fatty acid mixture.

In some embodiments, the fatty acids of the fatty acid mixtures of the compositions originate from, i.e. are isolated from, an oil of a natural source, e.g. a raw oil, such as from an oil from an aquatic animal or plant, a natural non-aquatic plant oil or a combination of such oils. Preferably, the fatty acids originate from an oil, or a combination of oils, from an aquatic animal or plant, such as from a marine or fresh water organism. More preferably, the fatty acids originate from a marine oil, i.e. an oil originating from a marine animal or plant. In one embodiment the natural oil is not derived from sponges, and the group of sponges is disclaimed from the group of natural sources. Oil from either of the marine and/or fresh water sponges are disclaimed. The marine oils may be selected from the list including, but not limited to, fish oil, mollusc oil, crustacean oil, sea mammal oil, plankton oil, algal oil and microalgal oil. The fatty acids of the fatty acid mixtures can also originate from a combination of two or more natural sources as described above. The term “fish oil” encompass all lipid fractions that are present in any fish species. “Fish” is a term that includes the bony fishes as well as the Chondrichthyes (cartilaginous fishes like sharks, rays, and ratfish), the Cyclostomata and the Agnatha. Without limiting the choice of raw materials, among the bony fishes preferred species can be found among fish of families such as Engraulidae, Carangidae, Clupeidae, Osmeridae, Salmonidae and Scombridae. Specific fish species from which such oil may be derived include herring, capelin, anchovy, mackerel, blue whiting, sand eel, cod and pollock. The oil can be derived from the whole fish, or from parts of the fish, such as the liver or the parts remaining after removing the fish fillets. Among the cartilaginous fish species, like sharks, the oil may preferably be obtained from the livers. The term “mollusc oil” includes all lipid fractions that are present in any species from the phylum Mollusca, including any animal of the class Cephalopoda, such as squid and octopus. The term “plankton oil” as utilised here, means all lipid fractions that can be obtained from the diverse collection of organisms that live in large bodies of water and are unable to swim against a current, not including large organisms such as jellyfish. The term “natural plant oils” is meant to include oil from algae and microalgae, and also meant to include oil from any single cell organisms. Thus, the natural plant oils may be selected from all oils derived from non-transgenic plants, vegetables, seeds, algae, microalgae and single cell organisms.

As employed herein, the terms “natural oil” and “oils from a natural source” and “raw oil” mean any fatty acid containing lipids, including, but not limited to one or more of glycerides, phospholipids, diacyl glyceryl ethers, wax esters, sterols, sterol esters, ceramides or sphingomyelins obtained from natural organisms. The natural organisms have not been genetically modified (non-GMO).

In nature the double bonds of fatty acids are all in the cis-form. In polyunsaturated omega-3 and omega-6 fatty acids each double bond is separated from the next by one methylene (—CH2-) group. The all cis-form as well as the exact position of the double bonds in the fatty acid molecule are vital for the biological transformations and actions of the fatty acids. The actions of the natural fatty acids in the body may set them apart from chemically synthesised fatty acids, which invariably contain some amounts of trans-isomers, as well as fatty acids where the position(s) of double bond(s) deviate from that of the beneficial natural fatty acids, all of which can result in biological actions competing with those of their natural counterparts. The VLCPUFAs of the fatty acids mixture of the invention are all on cis-form.

The fatty acids of the fatty acid mixtures and of the compositions of the invention have been isolated and concentrated from the natural source to obtain an enriched amount of the fatty acids. There is only a small amount of the VLCn3s present in natural oils like fish oils. As VLCUSFAs are only found naturally in extremely small quantities in a few organs of certain animal species, means for commercial production have been non-existent. Further, fatty acids with chain length above that of DHA, i.e. above C22, are usually removed in the processes for purifying fatty acids from marine oils, as higher molecular weight components are associated with undesirable constituents, such as oligomers and polymers formed from fatty acids, and also unsaponifiable constituents, such as cholesterol. Hence, when preparing enriched compositions of polyunsaturated fatty acids (LCPUFAs) from marine oils, the heavier VLCUSFAs have usually been removed and discarded, as a result of removing other heavy components.

The applicant has now found that VLCUSFAs can be prepared from natural sources such as from marine oils and provides such new compositions. One benefit is the improved and sustainable use of the raw material, as what was earlier considered to be a waste product from production of other fatty acid compositions, particularly from the preparation of compositions rich in EPA and DHA, can now be used to prepare a valuable VLCUSFA-comprising composition. The applicant has surprisingly found that it is possible to prepare the claimed composition, comprising both VLCPUFAs and VLCMUFAs, by isolating and concentrating (i.e. enrich) the VLCUFSAs from natural sources such as from marine oils, even though natural sources have a very low content of such fatty acids. Particularly, the applicant found that one can surprisingly selectively up-concentrate the VLC fatty acids by distillation. The VLC fatty acids can be separated from long chain fatty acids by distillation with surprising selectivity, enabling production of high concentrates of VLCMUFA and VLCPUFAs.

The overview below provides the approximate amounts of VLCUSFAs present in different examples of natural oils:

| Oil | VLC PUFAs (C24-C30) % | VLC MUFAs (C24-30) % |
|---|---|---|
| Sardine | 0.81 | 0.49 |
| Mackerel | 0.87 | 1.19 |
| Herring | 0.67 | 0.80 |
| Pollock | 0.66 | 0.57 |
| Blue whiting | 0.62 | 0.71 |
| Capelin | 0.36 | 0.77 |
| Farmed Salmon | 0.30 | 0.44 |
| Krill oil | 0.19 | 0.22 |
| Herring roe extract | 0.73 | 1.52 |

The above information has been obtained by applicant's analysis of the raw oils by gas chromatography (GC FID), and the results are given as area percentages (A %). The oils may also contain VLCFAs with chain lengths above C30.

Although the raw oils such as those above comprise a very low amount of VLCPUFAs and VLCMUFAs, compositions as claimed can be prepared from these, and the applicant has found that both VLCPUFAs and VLCMUFAs may be enriched from these oils.

The fatty acid compositions according to the present invention may typically be obtained and isolated by suitable procedures for transesterification or hydrolysis of the fatty acids from the natural oil, wherein the fatty acids are typically mainly on glyceride form, and subsequent physico-chemical purification processes. The fatty acids are not chemically synthesised. In one embodiment, the VLCUSFAs of the composition is unmodified as compared to the oil isolated from the natural source. Hence, in one embodiment, the chain length of the VLCPUFAs are unmodified, and preferably, the natural VLCUSFAs are included in the compositions, without any steps for elongations having taken place. Further, the compositions do not comprise any lipid producing cells that secrete or produce the VLCUSFAs. Rather, the compositions comprise a certain amount of VLCUSFAs, wherein these are isolated and up-concentrated from a natural source, using a method suitable for up-scaling and production for commercial use. Hence, the amount of the VLCUSFAs, including both VLCMUFAs and VLCPUFAs, has been increased, preferably considerably, compared to the content of the same fatty acids in the starting oil. The composition of the starting oil is of course decisive for what the composition of the end product is, although fractions from different process steps and from different starting oils may be combined to prepare the fatty acid mixtures of the compositions.

In one aspect of the invention, the fatty acid mixture of the composition comprises a reduced amount of cholesterol, compared to the content of the start oil. As higher molecular weight components of marine oils are typically associated with undesirable unsaponifiable constituents, including cholesterol, there is a particular need to separate the VLC fatty acids from cholesterol. Unexpectedly, the applicant has realized that VLCUSFAs can be isolated from oils that comprise e.g. cholesterol and various glycerides, and that the VLCUSFAs can be separated from the cholesterol and up-concentrated. The applicant has found that VLCPUFAs and VLCMUFAs are volatile enough to go as a distillate fraction using high quality molecular/short path distillation procedures without being thermally degraded, and provides a method for such procedure. Further, it was surprisingly found that the VLC fatty acids could be separated from glycerides and cholesterol esters by distillation enabling production of a fatty acid mixture with an enriched amount of VLC fatty acids combined with a reduced amount of cholesterol. The amount of cholesterol is measured as total cholesterol, i.e. cholesterol from free and esterified cholesterol (ref. Ph. Eur. Chapter 2.4.32; USP Omega-3 acid ethyl esters). In one embodiment, the fatty acid mixture of the composition comprises cholesterol in an amount less than 30 mg/g, such as less than 15 mg/g, such as less than 5.0 mg/g, such as less than 4.0 mg/g, such as less than 3.0 mg/g. Preferably, cholesterol is removed such that the amount of cholesterol present is close to zero, e.g. as low as 0.1 mg/g of the fatty acid mixture.

Particularly, in one embodiment the invention provides a composition comprising a fatty acid mixture wherein the fatty acid mixture comprises at least 1% by weight of very long chain monounsaturated fatty acids and at least 1% by weight of very long chain polyunsaturated fatty acids derived from natural oils, and where the fatty acid mixture contains less than 30 mg/g of cholesterol. More preferably, the fatty acid mixture of such composition comprises less than 5 mg/g cholesterol (mg cholesterol/g fatty acid mixture).

In another embodiment, the invention provides a composition comprising a fatty acid mixture wherein the fatty acid mixture comprises at least 0.5% by weight of very long chain monounsaturated fatty acids and at least 0.5% by weight of very long chain polyunsaturated fatty acids derived from natural oils, and where the fatty acid mixture contains less than 1.5 mg/g of cholesterol.

In one embodiment, the fatty acid mixture of the composition comprises at least 4% VLCMUFAs and at least 1% VLCPUFAs, as disclosed above, wherein the fatty acid mixture comprises less than 5 mg/g cholesterol. More preferably, such fatty acid mixture comprises at least 8% VLCMUFAs.

The fatty acid mixtures, particularly those with such a low-cholesterol content, preferably comprise fatty acids in an amount of at least 90.0%, 95.0% 97.0%, such as 98.0%, such as 99.0%, and preferably more than 99.5% fatty acids by weight. Hence, the fatty acid mixture is highly purified comprising substantially only fatty acids, comprising PUFAs and MUFAs as disclosed, such as omega-3 LCPUFAs, in addition to being enriched with VLCMUFAs and VLCPUFAs. The fatty acids can be provided in different forms, as later herein disclosed. The total weight % of unsaturated fatty acids, including long chain and very long chain PUFAs, is preferably at least 30%, such as at least 40%, more preferably at least 50%. In one embodiment, the fatty acid mixture comprises at least 30%, such as at least 40%, more preferably at least 50 weight % as the sum of mono- and polyunsaturated long chain fatty acids, in addition to the VLUSFAs present. In another embodiment, the sum of LC and VLC unsaturated fatty acids is at least 30 weight %.

The purified and up-concentrated fatty acid mixture of the invention further has a very low amount of unwanted pollutants. For instance, as shown in Example 7 (Table 12) and Example 9 (Table 19) below, compositions have been prepared wherein the amount of oligomeric and polymeric by-products, including oxidation products has been considerably reduced from the amount of such in the start oil. Preferably, such oxidation products are at maximum 1.5%, such as maximum 1.0%, more preferably at maximum 0.5% by weight of the fatty acid mixture. More particularly, the amount of environmental pollutants, like benzo(a)pyrene (BAP) and polyaromatic hydrocarbons (PAH), is low in the fatty acid mixture of the invention. In one embodiment, the fatty acid mixture of the composition comprises less than 2 μg/kg of benzo(a)pyrene (BAP). In another embodiment, the fatty acid mixture preferably comprises less than 10 μg/kg of polyaromatic hydrocarbons (4PAH). 4PAH is defined as the sum of benz(a)anthracene, chrysene, benzo(b)fluoranthenes and benzo(a)pyrene.

Further, the purified and up-concentrated fatty acid mixtures of the invention preferably have an appealing clear colour, e.g. such as a faint clear colour or a clear light yellow colour. To assess whether the prepared oil, i.e. the fatty acid mixture, has an acceptable colour, the Gardner colour scale may be used. In one embodiment, the prepared fatty acid mixture has a Colour Gardner below 9, such as below 8, more preferably below 7, and most preferably below 6, such as Colour Gardner at around 5, as provided in Example 9, Table 19 below. The Gardner Colour scale as used in this application is as specified in technical standard ASTM D 1544.

The fatty acids of the compositions, both the VLCUSFAs and other fatty acids of the compositions, can be in different forms. In one embodiment, fatty acids of the composition are in a form selected from the group of free fatty acids; fatty acid salts; mono-, di-, triglycerides; esters, such as ethyl esters; wax esters; 0-acetylated w-hydroxy fatty acids (OAHFAs); cholesteryl esters; ceramides; phospholipids and sphingomyelins; alone or in combination. Or, the fatty acids may be in any form that can be absorbed in the digestive tract, or that can be absorbed by a bodily surface after topical application. Preferably, the fatty acids are in the form of free fatty acids, fatty acid salts, ethyl esters, glycerides or wax esters. In one embodiment, the VLCUSFAs of the compositions comprising VLCMUFAS and VLCPUFAs wherein the carboxylic acid groups have been reduced to hydroxyl groups, I.e. fatty alcohols, are disclaimed. In one embodiment, VLCPUFA hydroxylated derivatives called elovanoids (ELVs) are disclaimed. When referring to weight % of fatty acids in the mixture, any of the above broadest defined forms of the fatty acids may be used as basis for the calculation. Further, the fatty acids of the composition, provided in any of the forms listed above, are preferably not connected to other active ingredients. Accordingly, the fatty acid mixture of the composition is a pure, unreacted, highly concentrated VLCUSFA mixture. However, the fatty acid end groups may have been modified from the original, such as e.g. from glycerides to esters.

In a particular embodiment, the VLCUSFAs of the compositions are not linked to any steroids, such as e.g. oestrogen.

The highly concentrated and purified fatty acid mixtures of the compositions comprise a certain amount of VLCUSFA, wherein the VLCUSFA have been isolated and up-concentrated (e.g. enriched) from a natural source, using a method suitable for up-scaling and production for commercial use. The process for preparing the fatty acid mixtures of the invention typically includes process steps such as e.g. a) purification steps to remove impurities or unwanted components, b) steps to increase stability and/or increase concentration, and/or c) chemical reaction steps in any order. Such purifications steps may e.g. include distillations, any of alkali refining/deacidification e.g. to remove free fatty acids and water-soluble impurities, degumming, bleaching to remove oxidation products and coloured components, and deodorization to remove volatile components causing taste and odour. The concentration steps may include any of extractions and urea complexation, in addition to e.g. distillations and chromatography. The chemical reaction steps are typically done to change the form of the fatty acid end groups, such as e.g. from glycerides to esters.

In a preferred embodiment, the enriched fatty acid mixtures of the compositions are obtained by a method of production comprising a series of distillations to select and up-concentrate the VLCUSFAs. Preferably, the VLCUSFAs are isolated by methods including short path/molecular distillations. More preferably, the method also include urea complexation steps. The applicant has been able to selectively concentrate the VLC fatty acids. The VLC fatty acids can be separated from LC fatty acids, e.g. like DHA, with surprisingly good selectivity, enabling production of high concentrates of VLCMUFAs and VLCPUFAs. One option is to use an oil wherein valuable long chain omega-3 fatty acids have already been separated out, hence using a residue fraction from the production of omega-3-concentrates. Accordingly, one potential procedure includes using a residue from the second step of a traditional two-step short path/molecular distillation procedure for the manufacture of omega-3-concentrates. Such residue typically represents a low value by-product from traditional processing. Thus, omega-3 acid concentrates are typically manufactured by a two-step short path distillation of ethylated marine oil wherein in the first step the content of ethyl esters of fatty acids with chain length up to C18 is reduced. In the second step the residue from the first step is passed through a distillation unit in order to isolate a distillate rich in omega-3 acids, particularly EPA and DHA. In the case of an ethyl ester concentrate this distillate may be the final product. If the final product is to be marketed as a triglyceride product, a further transesterification step with glycerol is required. The residue from such second distillation or subsequent distillations contains a high amount of partial glycerides and is enriched in cholesterol, i.e. the amount of cholesterol is higher than in the starting oil for the distillation steps. The commercial value of such residue is currently very low, as the fatty acids that have been regarded as valuable (mainly EPA and DHA) have been collected in the distillate stream. However, such residue will contain most of the VLCUSFAs of the original oil, in addition to that this may still include high concentrations of DHA and EPA. Surprisingly, it has now been found that a fatty acid mixture according to the present invention, that includes an enriched amount of VLCUSFAs, and preferably with a low content of cholesterol, can be provided from such residue.

In a preferred embodiment, the composition of VLCUSFAs, with a reduced content of cholesterol, is obtained by a process for preparing the fatty acid mixtures which comprises at least one step for cholesterol removal. Such process steps include a step wherein free cholesterol is converted to a cholesterol ester. Such a conversion is preferably done enzymatically, such as with a lipase, e.g. as shown in Example 1. Further, the process includes a step wherein the cholesterol ester is separated from the very long chain fatty acids esters. Such separation is preferably done by one or more distillations such as high quality molecular/short path distillation procedures.

Hence, in a further aspect the invention provides a method for production of a composition according to the first or second aspects. The method includes steps to prepare a composition comprising a fatty acid mixture, wherein the fatty acid mixture comprises both very long chain monounsaturated fatty acids (VLMUFAs) and very long chain polyunsaturated fatty acids (VLCPUFAs), and further wherein the amount of cholesterol in the fatty acid mixture is minimized. The prepared enriched compositions are composed of desired fatty adds which have been isolated and concentrated from the oil of a natural source and at the same time the obtained composition comprises an acceptable low amount of cholesterol, as disclosed in the aspects above.

Accordingly, the invention provides a method for production of a composition comprising a fatty acid mixture comprising both VLCPUFAs and VLCMUFAs in an enriched amount, wherein the fatty acid mixture is prepared from an oil material, the method comprising the steps of:
i) converting free cholesterol present in the oil material to cholesterol esters; and
ii) separating the cholesterol esters of step i) from very long chain fatty acid esters present in the oil material of step i).

The oil material is derived from a natural source, and this starting oil material for the method may be chosen from the oils of a natural source described for the first aspects. Preferably the oil material is a marine oil. In one embodiment, the oil material is an oil from a natural source which has been processed, i.e. it may have already gone through steps as disclosed in the above paragraphs, e.g. purification steps to remove impurities or unwanted components, steps to increase stability and/or increase concentration, and/or chemical reaction steps. In a preferred embodiment, the oil material is an ethylated marine oil. Hence, the fatty acids of the oil material are preferably mainly on the ethyl ester form. In one embodiment, the oil material is an oil wherein long chain omega-3 fatty acids have already been separated out, and more specifically the oil material is a residue from short path/molecular distillation procedure for the manufacture of omega-3-concentrates.

In step i) the oil material is brought in contact with an esterification catalyst, such as a lipase, converting the free cholesterol into cholesterol esters. Suitable lipases are preferably immobilized enzymes such as Lipozyme 435, Novozymes, but also non-immobilised enzymes may work, although a more difficult after-use recovery is foreseen. The reaction conditions, including temperature, pressure and reaction time, are selected based on normal operation conditions used when converting ethyl esters to triglycerides by use of the same enzyme. Typically, a temperature in the range of 50-90° C. and a pressure of 1-50 mbar is appropriate. During the reaction step i) the amount of free cholesterol is gradually reduced, as the free cholesterol is almost completely converted to cholesterol esters while at the same time the ethyl esters only to a limited degree are converted to glycerides, as shown in Examples 1 and 9. This very surprisingly shows that the lipase accepts free-cholesterol as the alcohol substrate in the enzymatic synthesis of cholesterol ester. Normally such transformations are done by cholesterol esterase enzymes. The process can also be carried out utilizing other relative amounts, and other sources, of suitable enzyme preparations, as well as utilizing other reaction conditions, including other reaction times and vacuums than described herein and in the examples, and/or by including additional procedures that can be utilized to bring the reaction to completion, including procedures for removing ethanol that is formed as a by-product during the transesterification reactions. When the reaction of step i) is completed the material is e.g. cooled and filtered before step ii).

In step ii) the oil material from step i) comprising cholesterol esters and fatty acid esters, is distilled to separate the VLCMUFAs and VLCPUFAs from the cholesterol esters. Such separation is preferably done by one or more distillations such as high quality molecular/short path distillation procedures. In one embodiment, a first distillation is performed at conditions wherein a substantial part of the cholesterol ester can be collected as a residue waste fraction.

Compared to the amount of fatty acid ethyl esters that was present before the enzyme treatment, only a limited amount is lost in the residue as di- and triglycerides. This may be attributed to the very surprising effect as stated above: the free cholesterol can be almost completely converted to cholesterol esters, while at the same time the ethyl esters only to a limited degree are converted to di- and tri-glycerides. While the conversion to di- and triglycerides is low, the amount appears to be sufficient to serve as a beneficial solubilisation fluid which keeps the cholesterol esters in solution in order to avoid deleterious precipitation on the heating surface of the short parth/molecular still, and in order to reduce evaporation of cholesterol esters. In the absence of such a solubilisation fluid, precipitated cholesterol esters would be detrimental to the oil flow and heat transfer on the heating surface. The marine fatty acid glyceride phase, rich in VLCFAs and cholesterol esters, could be subjected to a further reaction, such as a hydrolysis or an ethylation step, in order to make the VLCFAs available for separation from the cholesterol, for example by precipitation of the cholesterol by cooling of the ethyl ester solution, by distillation, or by other means known in the art, Alternatively, glyceride solutions of VLC fatty acids containing cholesterol esters can represent valuable products per se, for example as ingredients to feed for aquaculture, especially in feed to fry of farmed fish, and for farmed crustaceans.

As illustrated by Example 5, the present invention can be utilised to reduce the content of cholesterol below what is possible utilising existing methods for manufacturing marine fatty acid compositions with low levels of cholesterol.

The distillate from such first distillation, as described above for step ii), comprising VLCUSFAs, may be distilled one or more further times. The conditions for the second and subsequent distillations should be chosen to ensure that the VLCUSFAs are preferably mainly in one fraction, such as in the residues, while lighter fractions are removed. In one embodiment, a first distillation is run at a higher temperature than a second distillation.

An analysis of the fatty acid mixture of the distilled oils, after steps i) and ii), surprisingly showed that the VLC-PUFAs and VLCMUFAs can be distilled without being thermally degraded. It was also surprisingly found that the VLC fatty acids could be separated from glycerides and cholesterol ester by distillation. Short path/molecular distillation, as described herein, is normally regarded as only giving a limited degree of fractionation, as the maximum degree of separation that can be obtained from a single path through the still is regarded as one theoretical plate.

The compositions presently disclosed may, in addition to the fatty acid mixture, comprise at least one additive. The choice of such additives depends on several factors, including the intended use and administration form. Such additives may solubilize, suspend, thicken, dilute, emulsify, stabilize, preserve, protect, colour, flavour, and/or fashion active ingredients into an applicable and efficacious preparation, such that it may be safe, convenient, and/or otherwise acceptable for use. Examples of additives include, but are not limited to, solvents, carriers, viscosity modifiers, diluents, binders, sweeteners, aromas, pH modifiers, antioxidants, extenders, humectants, disintegrating agents, solution-retarding agents, absorption accelerators, wetting agents, absorbents, lubricants, colouring agents, pigments, thickeners, stabilisers, glossing agents, gelling agents, dispersing agents, salts, oils, waxes, polymers, silicone compounds, biogenic agents, film formers, tonicity agents, emulsifiers, surfactants, buffers, inorganic and organic sunscreens, anti-inflammatory agents, free radical scavengers, moisturizers, vitamins, enzymes, and preservatives. Additives may have more than one role or function, or may be classified in more than one group; classifications are descriptive only and are not intended to be limiting. In some embodiments, for example, the at least one additive may be chosen from corn starch, lactose, glucose, microcrystalline cellulose, magnesium stearate, polyvinylpyrrolidone, citric acid, tartaric acid, water, ethanol, glycerol, sorbitol, polyethylene glycol, propylene glycol, cetylstearyl alcohol, carboxymethylcellulose, and fatty substances such as hard fat or suitable mixtures thereof. In some embodiments, the compositions presently disclosed comprise an antioxidant, chosen from the group including but not limited to tocopherol such as alpha-tocopherol, beta-tocopherol, gamma-tocopherol, and delta-tocopherol, or mixtures thereof, BHA such as 2-tert-butyl-4-hydroxyanisole and 3-tert-butyl-4-hydroxyanisole, or mixtures thereof and BHT (3,5-di-tert-butyl-4-hydroxytoluene), and ascorbyl palmitate or mixtures thereof.

In one aspect, the present invention is directed to the described fatty acid compositions, or any formulations comprising any of the described fatty acid compositions, for use as a medicament/pharmaceutical, nutraceutical composition, food supplement, food additive, or cosmetic product. In one embodiment, the disclosed composition is a pharmaceutical composition comprising any of the disclosed fatty acid mixtures. The pharmaceutical composition may also comprise one or more additional active pharmaceutical ingredients, and/or pharmaceutically acceptable carriers, excipients, and/or antioxidants. The pharmaceutical composition may be formulated for any conventional administration form, including but not limited to tablets, coated tablets, capsules, powders, granulates, solutions, dispersions, suspensions, syrups, creams, lotions, salves, gels, emulsions, sprays, suppositories, and pessaries. Conventional formulation techniques may be used. The compositions may be administered by any administration route, including but not limited to orally, intravenously, intramuscularly, sublingually, subcutaneously, intrathecally, buccally, rectally, vaginally, occularly, nasally, by inhalation, transdermally, or cutaneously.

In another embodiment, the present invention is directed to a food supplement, a food additive, or a nutraceutical preparation comprising any of the described fatty acid compositions. Such a food supplement, food additive or nutraceutical composition may be produced for administration through any route, including but not limited to as a liquid nutritional, as a foodstuff, and as a beverage. In one embodiment, the composition is for therapeutic use. For use in a food supplement, a food additive, or a nutraceutical preparation the composition may be in the form of capsules, preferably gelatine capsules, and the capsule may be flavoured; tablets, powders or liquids.

In yet another embodiment, the present invention is directed to a cosmetic formulation comprising the fatty acid composition disclosed, such as in a cosmetic dermatological product. Such a cosmetic formulation may be chosen from the group including, but not limited to, powders, solutions, dispersions, suspensions, creams, lotions, salves, gels, emulsions, sprays, pastes, sprays, solids and semi-solids. The cosmetic formulation may be applied using any known method for application, to skin, mucous membranes, nails and/or hair.

The VLCUSFAs appear to have a role in upholding barriers between the human or animal body surfaces and the environment, including the skin and/or lung and/or intestinal barriers. This includes the body's barrier functions towards moisture, particularly in order to avoid drying out of the body, protection against drying/wrinkling of the skin and photo-ageing harming of the skin caused by UV-radiation, and further protection against pathogen microorganisms entering into the body. In one embodiment, the compositions of the invention are for use in protection of the skin against photo-ageing. In another embodiment, the compositions of the invention are for use in improving the skin's barrier against drying and against microorganism invasion.

It is to be understood that embodiments disclosed for one aspect also apply to other aspects of the invention. For instance, the embodiments disclosed for the compositions also apply for the aspect directed to the method for production.

It is to be understood that each component, compound, substituent, or parameter disclosed herein is to be interpreted as being disclosed for use alone or in combination with one or more of each and every other component, compound, substituent, or parameter disclosed herein.

It is also to be understood that each amount/value or range of amounts/values for each component, compound, substituent, or parameter disclosed herein is to be interpreted as also being disclosed in combination with each amount/value or range of amounts/values disclosed for any other component(s), compounds(s), substituent(s), or parameter(s) disclosed herein and that any combination of amounts/values or ranges of amounts/values for two or more component(s), compounds(s), substituent(s), or parameters disclosed herein are thus also disclosed in combination with each other for the purposes of this description.

It is further understood that each lower limit of each range disclosed herein is to be interpreted as disclosed in combination with each upper limit of each range disclosed herein for the same component, compounds, substituent, or parameter. Thus, a disclosure of two ranges is to be interpreted as a disclosure of four ranges derived by combining each lower limit of each range with each upper limit of each range. A disclosure of three ranges is to be interpreted as a disclosure of nine ranges derived by combining each lower limit of each range with each upper limit of each range, etc.

Specific embodiments of the invention are listed below.
1. A composition comprising a fatty acid mixture wherein the fatty acid mixture comprises at least 1% by weight of very long chain monounsaturated fatty acids and at least 1% by weight of very long chain polyunsaturated fatty acids derived from natural oils, and where the fatty acid mixture contains less than 30 mg/g of cholesterol, such as less than 5 mg/g cholesterol.
2. A composition comprising a fatty acid mixture wherein the fatty acid mixture comprises at least 0.5% by weight of very long chain monounsaturated fatty acids and at least 0.5% by weight of very long chain polyunsaturated fatty acids derived from natural oils, and where the fatty acid mixture contains less than 1.5 mg/g of cholesterol.
3. A composition comprising a fatty acid mixture wherein the fatty acid mixture comprises at least 1% by weight of very long chain monounsaturated fatty acids and at least 1% by weight of very long chain polyunsaturated fatty acids derived from natural oils, and where the fatty acid mixture further comprises at least 10% by weight of C20-C22 monounsaturated fatty acids.
4. The composition of any of the items 1-3 wherein the fatty acid mixture comprises at least 2% by weight of very long chain monounsaturated fatty acids.
5. A composition comprising a fatty acid mixture wherein the fatty acid mixture comprises at least 4%, such as at least 8% by weight of very long chain monounsaturated fatty acids and at least 1% by weight of very long chain polyunsaturated fatty acids derived from natural oils.
6. The composition of any of the items 1-5 wherein the natural oils are oils from marine or fresh water organisms.
7. The composition of any of the items 1-6 wherein the natural oil is selected from the group of fish oil, mollusc oil, crustacean oil, sea mammal oil, plankton oil, algal oil and microalgal oil.
8. The composition of any of the items 1-7 wherein the fatty acid mixture comprises at least 15% by weight of very long chain monounsaturated fatty acids.
9. The composition of any of the items 1-8 wherein the fatty acid mixture comprises at least 1% by weight of very long chain monounsaturated fatty acids with chain length of more than 24 carbon atoms.
10. The composition of any of the items 1-9 wherein the fatty acid mixture comprises at least 6% by weight of very long chain monounsaturated fatty acids with chain length of more than 24 carbon atoms.
11. The composition of any of the items 1-9 wherein the fatty acid mixture comprises at least 10% by weight of very long chain monounsaturated fatty acids with chain length of more than 24 carbon atoms.
12. The composition of any of the items 1-11, wherein the fatty acid mixture comprises at least 2% by weight of one or more very long chain polyunsaturated fatty acids.
13. The composition of any of the items 1-12, wherein the fatty acid mixture comprises at least 5% by weight of one or more very long chain polyunsaturated fatty acids.
14. The composition of any of the items 1-13, wherein the fatty acid mixture comprises at least 10% by weight of one or more very long chain polyunsaturated fatty acids.
15. The composition of any of the items 1-14, wherein the fatty acid mixture comprises at least 10% by weight of one or more very long chain omega-3 polyunsaturated fatty acids.
16. The composition of any of the items 1-15 wherein the fatty acid mixture comprises very long chain monounsaturated fatty acids and very long chain polyunsaturated fatty acids in a total amount of at least 20%.
17. The composition of any of the items 1-16 wherein the fatty acid mixture comprises very long chain monounsaturated fatty acids and very long chain polyunsaturated fatty acids in a total amount of at least 50%.
18. The composition of any of the items 1-17 wherein the fatty acid mixture comprises very long chain monounsaturated fatty acids and very long chain omega-3 polyunsaturated fatty acids in a total amount of at least 50%.
19. The composition of any of the items 1-18 wherein the fatty acid mixture comprises very long chain monounsaturated fatty acids and very long chain omega-3 polyunsaturated fatty acids in the weight ratio 3:1-1:2.
20. The composition of any of the items 1-19, wherein the fatty acid mixture comprises at least 5% by weight of one or more C28 very long chain polyunsaturated fatty acids.
21. The composition of any of the items 1-20, wherein the fatty acid mixture comprises at least 5% by weight by weight of at least one of the very long chain fatty acids C28:6n3 and C28:8n3.
22. The composition of any of the items 1-21, wherein the fatty acid mixture comprises at least 5% by weight of the very long chain fatty acid C26:6n3.
23. The composition of any of the items 1-22, wherein the fatty acid mixture comprises at least 5% by weight of the very long chain fatty acid C24:5n3.
24. The composition of any of the items 1-23, wherein the fatty acid mixture further comprises at least 1% by weight of C18-C22 monounsaturated fatty acids.
25. The composition of any of the items 1-24, wherein the fatty acid mixture further comprises at least 1% by weight of C20-C22 polyunsaturated fatty acids (LCPUFAs), such as at least 5% LCPUFAs, such as at least 10%, at least 20%, at least 25%, at least 30%, at least 40%, at least 50%, or at least 60%.
26. The composition of any of the items 1-25 wherein said fatty acids are in the form of free fatty acids, free fatty acid salts, mono-, di-, triglycerides, ethyl esters, wax esters, cholesteryl esters, ceramides, phospholipids or sphingomyelins, alone or in combination.
27. The composition of any of the items 1-26 wherein said fatty acids are in the form of free fatty acids, free fatty acid salts, ethyl esters, glycerides or wax esters.
28. The composition of any of the items 1 and 3-27 wherein the fatty acid mixture comprises less than 5 mg/g cholesterol.
29. The composition of any of the items 1-28 wherein the fatty acid mixture does not contain acetylenic fatty acids.

### EXAMPLES

The examples below are provided to illustrate that compositions of VLCUSFAs as claimed can be prepared from natural oils, wherein the fatty acids derive from natural oils and the amount of these very long chain fatty acids has been enriched. The examples show that different VLCMUFAs and VLCPUFAs can be up-concentrated, that the fatty acids can be provided in different forms, that the compositions have a high purity, and that VLCUSFAs can be separated from the cholesterol and up-concentrated. The examples further show that a residue fraction from the production of long-chain omega-3-concentrates (typically including EPA and DHA), can be used to prepare the claimed VLCUASFAs compositions, providing a sustainable use of the raw oil.

In the following examples, oils from mackerel or sardine were used, as this was available to the applicant. Similar processes and examples could equally well have been performed using other oils comprising some VLCUSFAs, such as oils from a marine or fresh water organism. For example, oils from herring, pollock, blue whiting, capelin, farmed salmon, krill oil, or herring roe extract could have been used as the starting oil.

### Example 1: VLCUSFA Composition from Sardine and Mackerel Oil

120 kg of a residue, from a commercial scale distillation of an ethylated sardine and mackerel oil utilized to produce an omega-3-acid concentrate containing about 36% EPA and about 25% DHA, was converted to ethyl esters by reacting it with 25 w % (of oil weight) of 2% sodium ethoxide in ethanol. The mixture was stirred for 1 hour at 80° C. The excess ethanol was then evaporated under vacuum. The stirring was stopped, and after 30 minutes a small dark heavy phase of glycerol was drained out of the reactor via the bottom valve. The oil was then washed with water containing 5% citric acid and twice with water. The oil phase was then dried under vacuum at 40-50° C., providing 110 kg of an oil with the composition shown in Column 2 of Table 1.

The oil (column 2, Table 1) was taken through a double distillation in a short path distillation (VTA, model VK83-6-SKR-G with degasser). The temperature of the first column was 175° C. (flow of 4 kg/h and a vacuum of 0.01 mbar). The residue was collected as waste (10 kg) while the distillate was taken to the second column. The temperature of the second column was 130° C. (flow of approximately 3.2 kg/h and a vacuum of 0.01 mbar). The distillate (15 kg) was enriched in short chain fatty acids, while the purified product (85 kg) containing VLCFAs was collected as the oil residue (column 3, Table 1).

An analysis of glyceride content, free cholesterol content and fatty acid profile was performed for the start oil (column 2, Table 1), and the oil residue after the double distillation (column 3, Table 1).

TABLE 1
Fatty acid profile of different fractions during purification and up-concentration
of VLCFAs. Results are given as area percentages (A %); in chromatograms from size
exclusion chromatography (SEC) for ethyl esters (EE), monoglycerides (MG), diglycerides
(DG) and triglycerides (TG), and as A % from gas chromatography (GC) for the
fatty acid analyses.
Column 2: Start oil
Column 3: Residue after double distillation
Column 4: Residue from double distillation of enzymatic treated oil of column 3
Column 5: Residue from distillation of the oil in column 4
Column 6: Distillate from distillation of the oil in column 5

| Column 1 | 2 | 3 | 4 | 5 | 6 |
|---|---|---|---|---|---|
| | Start | D175R130 | Enzym + | R | D |
| | | | D180R130 | | |
| EE (%) | 52.5 | 78.4 | | | |
| MG (%) | 23.2 | 21.54 | | | |
| DG (%) | 17.6 | 0 | | | |
| TG (%) | 6.5 | 0 | | | |
| Free cholesterol | NA | 41.1 | | | |
| (mg/g) | | | | | |
| Total cholesterol | 52.50 | >41.1 | 0.6 | 0.3 | |
| (mg/g) | | | | | |
| Fatty acid | A % GC | A % GC | A % GC | A % GC | A % GC |
| C14:0 | 1.46 | 0.04 | | | 0.31 |
| C16:0 | 4.74 | 0.33 | | | 0.45 |
| C16:1 | 1.91 | 0.1 | | | 0.2 |
| C16:4 | 0.3 | 0.03 | | | 0.04 |
| C18:0 | 1.58 | 0.68 | 0.13 | | 0.42 |
| C18:1n9 | 2.74 | 0.89 | 0.13 | | 0.49 |
| C18:1n7 | 1.11 | 0.35 | 0.06 | | 0.19 |
| C18:2n6 | 0.39 | 0.12 | | | 0.07 |
| C18:3n3 | 0.28 | 0.09 | | | 0.05 |
| C18:4n3 | 0.74 | 0.07 | 0.03 | | 0.14 |
| C20:1 | 1.7 | 1.4 | 0.89 | 0.29 | 1.96 |
| C20:4n6 | 1.17 | 0.84 | 0.41 | 0.09 | 1.01 |
| C20:4n3 | 0.89 | 0.84 | 0.5 | 0.14 | 1.17 |
| C20:5n3 | 14.44 | 11.22 | 5.56 | 1.15 | 13.92 |
| C22:0 | 0.49 | 0.93 | 1.1 | 1.29 | 0.62 |
| C22:1 | 6.16 | 8.59 | 9.63 | 10.1 | 7.67 |
| C21:5n3 | 0.76 | 8.59 | 1.54 | 1.66 | 1.12 |
| C22:5n6 | 1.11 | 0.97 | 1.01 | 0.99 | 1.74 |
| C22:5n3 | 6.96 | 8.64 | 9.3 | 8.37 | 9.73 |
| C22:6n3 | 32.57 | 39.45 | 42.33 | 35.72 | 48.99 |
| C24:0 | 0.38 | | 1.11 | 1.78 | 0.17 |
| C24:1 | 4.02 | 6.55 | 8.86 | 14.16 | 1.05 |
| C24:1 | 0.05 | 0.58 | 0.76 | 1.15 | 0.21 |
| C24:2 | 0.02 | 0.04 | 0.08 | 0.23 | |
| C24:4n3 | 0.08 | 0.12 | 0.23 | 0.34 | |
| C24:5n3 | 0.91 | 1.31 | 1.79 | 2.69 | 0.38 |
| C26:0 | | | 0.05 | 0.14 | |
| C24:6 | 0.39 | 0.59 | 0.79 | 1.21 | 0.16 |
| C26:1 | 0.71 | 1.11 | 1.41 | 2.61 | 0.02 |
| C26:3 | 0.07 | 0.11 | 0.14 | 0.25 | |
| C26:4n3 | 0.17 | 0.24 | 0.31 | 0.56 | 0.01 |
| C26:5n3 | 0.24 | 0.36 | 0.48 | 0.89 | |
| C26:6n3 | 0.48 | 0.69 | 0.97 | 1.73 | 0.04 |
| C26:7n3 | | 0.08 | 0.11 | 0.21 | 0.01 |
| C28:0 | | 0.06 | 0.02 | 0.12 | |
| C28:1 | 0.07 | | 0.02 | 0.04 | |
| C28:2 | 0.05 | 0.02 | 0.06 | 0.11 | |
| C28:4n3 | 0.11 | 0.13 | 0.08 | 0.16 | |
| C28:5n3 | 0.04 | 0.06 | 0.08 | 0.14 | |
| C28:6n3 | 0.13 | 0.2 | 0.22 | 0.42 | 0.01 |
| C28:7n3 | 0.03 | 0.03 | 0.05 | 0.08 | |
| C28:8n3 | 1.04 | 1.48 | 1.86 | 3.51 | 0.02 |
| C30:5n3 | | | | | |
| C30:6n3 | | 0.07 | 0.03 | 0.06 | |
| C30:8n3 | | 0.07 | 0.05 | 0.09 | |
| C32 | 0 | 0.05 | 0.05 | 0.1 | |
| Sum VLCPUFA | 3.69 | 5.54 | 7.24 | 12.48 | 0.63 |
| VLCMUFA | 4.85 | 8.24 | 11.05 | 17.96 | 1.28 |

### Enzymatic Treatment:

The oil residue from the double distillation (82.7 kg) (column 3, Table 1) was added 1.93 kg of Immobilised enzyme (Lipozyme 435, Novozymes), and the mixture was stirred at 80° C. and vacuum (10 mbar) for 46 hours. After cooling and filtration, the oil was taken to distillation.

The results from the analysis of samples during enzymatic treatment are shown below in Table 2.

TABLE 2

| Column 1 | 2 | 3 | 4 | 5 | 6 |
|---|---|---|---|---|---|
| Time | Start | 5 h | 23 h | 30 h | 46 h |
| Free | 41.11 | 22.38 | 3.03 | 0.95 | 0.25 |
| Cholesterol | | | | | |
| mg/g | | | | | |
| EE (%) | 78.46 | 71.38 | 64.36 | 63.47 | NA |
| MG (%) | 21.54 | 17.09 | 16.90 | 16.41 | NA |
| DG (%) | 0 | 3.64 | 10.19 | 10.82 | NA |
| TG (%) | 0 | 7.89 | 8.85 | 9.29 | NA |

Table 2 shows that the free cholesterol is gradually reduced from 41.11 mg/g to 0.25 mg/g after 46 h reaction time. This means that during the enzymatic step the free cholesterol is converted to cholesterol esters. This surprisingly shows that the Lipase accept free-cholesterol as the alcohol substrate in the enzymatic synthesis of cholesterol ester. Normally such transformations are done by cholesterol esterase enzymes.

The process described above can also be carried out utilizing other relative amounts, and other sources, of suitable enzyme preparations, as well as utilizing other reaction conditions, including other reaction times and vacuums than described in this example, and/or by including additional procedures that can be utilized to bring the reaction to completion, including procedures for removing ethanol that is formed as a by-product during the transesterification reactions.

During the reaction, the amount of monoglycerides (MG) was reduced, and amounts of di- and triglycerides (DG, TG) were increased. It should be noted that the size-exclusion chromatography (SEC) method, which is similar to that described in the European Pharmacopoeia and USP monographs for omega-3-acid triglycerides, omega-3-acid ethyl esters and fish oils, probably overestimates the MG content and underestimate the ethyl ester (EE) content in samples high in long chain fatty acids, as the long chain EEs will have similar molecular size as shorter chain MGs, and for this reason partly coelute with the MGs. Therefore, the real content of MG in the sample after 46 hours is probably low.

The enzyme treated oil, after 46 hours (column 6, Table 2), was then taken through a double distillation in a short path distillation (VTA, model VK83-6-SKR-G with degasser). The temperature of the first column was 180° C. (flow of 4 kg/h and a vacuum of 0.01 mbar), the residue was collected as waste while the distillate was taken to the second column. The temperature of the second column was 130° C. (flow of approximately 3.3 kg/h and a vacuum of 0.01 mbar). The distillate from the second column (20.8 kg) was enriched in shorter chain fatty acids, while the purified product (43.2 kg) containing enriched amounts of VLC-unsaturated fatty acids was collected as the residue from the second column (column 4, Table 1). The total cholesterol of this composition was only 0.6 mg/g, being reduced from 52.5 mg/g in the starting oil (column 2, Table 1). This very substantial reduction in total cholesterol was caused by the cholesterol esters being removed in the residue fraction from the first of the distillation steps as described above.

The residue oil from the second distillation after enzymatic treatment (column 4, Table 1) was then taken through a series of distillations in a short path distillation apparatus (temperature 130-141° C. and vacuum 0.01 mbar, VTA, model VK83-6-SKR-G with degasser). For each step a light fraction (20-30%) was removed as a distillate while the residue was taken back to the next distillation steps. The composition of the residue (R) from the first distillation step is shown in column 5 in Table 1. Column 6, Table 1 shows a typical composition of the distillate (D). The compositions of the residues from the following distillations (RR-RRRRRR) are shown in column 2-6 in Table 3.

TABLE 3
Fatty acid profile from distillations 2-6.

| Column 1 | 2 | 3 | 4 | 5 | 6 |
|---|---|---|---|---|---|
| | RR | RRR | RRRR | RRRRR | RRRRRR |
| Fatty acid | | | | | |
| C20:1 | 0.17 | | 0.03 | | |
| C20:4n6 | 0.04 | | | 0.09 | |
| C20:4n3 | 0.06 | | | | |
| C20:5n3 | 0.71 | 0.18 | 0.13 | 1.25 | 1.11 |
| | 1.34 | | | 0 | |
| C22:1 | 10.23 | 9.84 | 1.39 | 6.35 | 5.07 |
| C21:5n3 | 1.63 | 1.67 | 8.92 | 1.18 | 0.96 |
| C22:5n6 | 1.05 | 0.57 | 0.94 | 1.25 | 0.19 |
| C22:5n3 | 7.85 | 6.81 | 5.69 | 2.84 | 2.1 |
| C22:6n3 | 32.61 | 26.93 | 21.98 | 10.63 | 8.06 |
| C24:0 | 2 | 2.34 | 2.67 | 3.56 | 3.73 |
| C24:1 | 16.37 | 19.17 | 21.55 | 28.58 | 29.24 |
| C24:1 | 1.3 | 1.5 | 1.68 | 2.2 | 2.25 |
| C24:2 | 0.25 | 0.29 | | 0.5 | 0.43 |
| | | | | 0.51 | 0.53 |
| C24:4n3 | 0.39 | 0.45 | 0.53 | 0.67 | 0.68 |
| C24:5n3 | 2.98 | 3.46 | 3.8 | 4.48 | 4.58 |
| | 0.15 | | | 0.36 | 0.42 |
| C24:6n3 | 1.35 | 1.59 | 1.81 | 2.12 | 2.22 |
| C26:0 | | | | | |
| C26:1 | 3.04 | 3.69 | 4.42 | 6.22 | 6.82 |
| C26:2 | 0.28 | 0.04 | 0.4 | 0.56 | |
| C26:4n3 | 0.65 | 0.78 | 0.93 | 1.3 | 1.43 |
| C26:5n3 | 1.01 | 1.2 | 1.44 | 1.99 | 2.22 |
| C26:6n3 | 1.98 | 2.36 | 2.79 | 3.82 | 4.2 |
| C26:7n3 | 0.24 | 0.28 | 0.34 | 0.45 | 0.48 |
| C28:0 | 0.12 | 0.15 | 0.16 | 0.16 | 0.18 |
| C28:1 | 0.04 | 0.06 | 0.05 | 0.1 | |
| C28:2 | 0.13 | 0.17 | 0.21 | 0.26 | 0.32 |
| C28:4n3 | 0.19 | 0.27 | 0.35 | 0.43 | 0.55 |
| C28:5n3 | 0.16 | 0.28 | 0.35 | 0.44 | 0.53 |
| C28:6n3 | 0.48 | 0.62 | 0.77 | 0.98 | 1.2 |
| C28:7n3 | 0.1 | 0.12 | 0.14 | 0.19 | 0.23 |
| C28:8n3 | 3.96 | 4.88 | 6.04 | 8.02 | 9.39 |
| C30:5n3 | | | | | |
| C30:6n3 | 0.06 | 0.09 | 0.15 | 0.13 | 0.16 |
| C30:8n3 | 0.09 | 0.13 | 0.18 | 0.17 | 0.22 |
| | | | | | 0.22 |
| C32 | 0.1 | 0.15 | 0.16 | 0.23 | 0.3 |
| Sum | 14.14 | 16.55 | 20.02 | 27.07 | 29.34 |
| VLCPUFA | | | | | |
| Sum | 20.75 | 24.42 | 27.7 | | 38.31 |
| VLCMUFA | | | | | |
| Sum | 35.15 | 41.58 | 48.09 | 64.59 | 68.2 |
| VLCUSFA | | | | | |

An analysis of the distilled oils, both after ethylation, after enzyme treatment, and distillations surprisingly showed that the VLC-PUFAs and VLCMUFAs can be distilled without being thermally degraded. It was also surprisingly found that the VLC fatty acids could be separated from glycerides and cholesterol ester by distillation. Short path/molecular distillation, as described here, is normally regarded as only giving a limited degree of fractionation, the maximum degree of separation that can be obtained from a single path through the still is regarded as one theoretical plate.

The distillation steps described above show that one surprisingly can selectively up concentrate the VLC fatty acids. The VLC fatty acids can be separated from LC fatty acids, like DHA, with surprising selectivity, enabling production of high concentrates of VLCMUFA and VLCPUFAs.

### Urea Fractionation:

Some of the oil from the last distillation (Column 6, Table 3) was taken through a urea precipitation procedure. Urea fractionation is a method for isolating separate fractions of fatty acids having identical chain lengths but different degrees of unsaturation.

225 g of urea was mixed with 450 g of ethanol (96%) in a jacketed reactor and heated to 80° C. under stirring. 150 g of oil (column 6, Table 3) was added, and the mixture stirred for 30 minutes. After cooling to 25° C. the mixture was filtered, the ethanol of the filtrate was partially evaporated, and the mixture filtered a second time. The oil was then washed with 5% citric acid in water and twice with water and dried under vacuum to yield 73.9 g of oil (column 2, Table 4). This product oil (35 g) was distilled using a short path distillation still (VTA, model VKL-70-4-SKR-T). After a first distillation at a temperature of 132° C., a flow of 3.5 ml/min and a pressure of 10⁻³ mbar, a residue (˜10 g) (“R132”, Column 4, Table 4) and a distillate (25 g) were collected. A second portion of the same product oil (˜35 g) was also distilled at 135° C., a flow of 3.5 ml/min and a pressure of 10⁻³ mbar, and a residue (˜8 g)(“R135”, Column 5) and a distillate (˜27 g) were collected. The two distillates from the distillations at 132 and 135° C. (˜52 g) were combined and distilled at temperatures of 126° C. and 130° C., respectively (˜25 g each), at a flow of 3.5 ml/min and a pressure of 10⁻³ mbar. The compositions of the residues from the distillations at 126° C. (˜8 g) and 130° C. (˜7 g) are given in column 6 (“DR126”) and 7 (“DR130”), (Table 4) respectively. Finally, the distillates (˜37 g) from distillations at 126 and 132° C. were combined and distilled at 122° C., the composition of the residue (“DDR122”) (˜7 g) is given in column 8, Table 4.

Some of the urea adduct from the urea precipitation was mixed with water and heptane. After phase separation, the heptane phase was washed twice with water and evaporated. The fatty acid composition of the urea adduct is shown in column 3 of Table 4.

TABLE 4

| Column 1 | 2 | 3 | 4 | 5 | 6 | 7 | 8 |
|---|---|---|---|---|---|---|---|
| | Urea | Urea | R132 | R135 | DR126 | DR130 | DDR122 |
| | 150% | Adduct | | | | | |
| Fatty acids | | | | | | | |
| C20:5 n3 | 0.45 | | 1.3 | 0.98 | 0.11 | 0.63 | |
| C22:0 | 0 | 2.19 | | | | | |
| C22:1 | 2.8 | 5.42 | 0.8 | 0.97 | 0.2 | 0.85 | 0.27 |
| C21:5 n3 | 0.33 | 1.29 | 0 | 0.15 | | 0.11 | |
| C22:5 n6 | 0.68 | 1.66 | 0 | 0.13 | | 0.14 | |
| C22:5 n3 | 4.99 | 0.08 | 0.71 | 0.92 | 0.16 | | 0.47 |
| C22:6 n3 | 14.95 | 0.92 | 2.83 | 3.13 | 0.62 | 1.01 | 1.37 |
| C24:0 | 0.05 | 8.01 | 0.71 | | | | |
| C24:1 | 1.45 | 50.15 | 1.24 | 1.45 | 0.93 | 1.25 | 1.28 |
| C24:1 | 0.11 | 3.77 | 0 | 0.07 | 0.01 | 0.11 | 0.13 |
| | | 0.63 | | 0.62 | 0.5 | 0.9 | 1.13 |
| C24:2 | 0.34 | 1 | 0.24 | 0.15 | 0.1 | 0.2 | 0.34 |
| C24:3 | 0.78 | 0.28 | | 0.42 | 0.36 | 0.61 | 0.79 |
| C24:4n3 | 1.43 | 0.06 | 0.29 | 0.61 | 0.48 | 0.94 | 1.19 |
| C24:5n3 | 10.7 | 0.24 | 1.46 | 3.78 | 2.59 | 5.98 | 7.26 |
| C26:0 | 0 | 0.77 | 0.4 | | | | |
| C24:6n3 | 0.44 | 0.37 | 0.4 | 1.118 | 0.73 | 1.85 | 2.13 |
| C26:1 | 0 | 12.84 | 0.21 | 0.22 | 0.13 | 0.07 | |
| C26:3 | 0.58 | 0.44 | 0.63 | 0.69 | 0.8 | 0.83 | 0.98 |
| C26:4n3 | 2.62 | 0.33 | 3.32 | 3.54 | 4.32 | 3.79 | 4.86 |
| C26:5n3 | 4.84 | 0.25 | 4.95 | 5.66 | 6.68 | 6.48 | 8.61 |
| C26:6n3 | 9.89 | 0.17 | 8.03 | 9.89 | 11.37 | 11.96 | 16.74 |
| C26:7n3 | 1.18 | | 0.76 | 1 | 1.11 | 1.27 | 1.8 |
| C28:0 | 0.42 | 0.05 | 0.59 | 0.57 | 0.64 | 0.56 | 0.57 |
| C28:1 | 0.24 | 0.03 | 0.44 | 0.42 | 0.49 | 0.4 | 0.44 |
| C28:4n3 | 1.02 | 0.1 | 3.79 | 2.96 | 3.01 | 2.33 | 1.08 |
| C28:5n3 | 1.16 | 0.01 | 2.37 | 2.24 | 2.54 | 1.98 | 1.45 |
| C28:6n3 | 5.67 | 0.06 | 7.18 | 5.98 | 6.31 | 4.91 | 3.9 |
| C28:7n3 | 0.52 | 0.01 | 1.23 | 1.03 | 1.15 | 0.9 | 0.82 |
| C28:8n3 | 21.56 | 0.31 | 46.96 | 40.82 | 46.47 | 37.41 | 35.6 |
| C30:6n3 | 0.28 | | 1.27 | 0.69 | 0.41 | 0.41 | 0.15 |
| C30:8n3 | 0.43 | 0.01 | 2.09 | 1.21 | 0.81 | 0.77 | 0.3 |
| | 0.03 | | 0.24 | 0.15 | | | 0.05 |
| C32n3 | 0.6 | 0.01 | 2.41 | 1.55 | 1.2 | 1.05 | 0.42 |
| Sum | 64.23 | 3.43 | 87.54 | 83.188 | 90.85 | 84.11 | 88.08 |
| VLCPUFA | | | | | | | |
| VLCMUFA | 1.98 | 66.81 | 2.04 | 2.31 | 1.71 | 1.99 | 1.98 |

The results show that VLCMUFAs can be effectively removed by urea precipitation, separating these from the VLCPUFAs, reducing the VLCMUFA content from 38.31% (column 6, Table 3) to 1.98% (column 2, Table 4), the content of VLCPUFA was at the same time increased from 29.34% to 64.23% (Column 6, Table 3). Analysis of the urea adduct showed a high content of VLCMUFA of 66.81% (Column 3, Table 4) and only 3.43% VLCPUFA.

Urea fractionation may hence be effectively employed to achieve isolated fractions of fatty acids within each group of VLCUSFAs with identical chain length. Thus, by using urea as a fractionation tool, the relative content of the most unsaturated VLCPUFAs within each chain length may be increased in the non-urea complexing fraction, while the relative content of the less unsaturated VLCPUFAs at the same time may be increased in the urea complexing fraction of the fatty acids. Thus, for example, the fatty acids with the lowest number of double bonds (starting with VLCMUFAs) can be step-wise isolated from a mixture of VLCPUFAs as urea adducts (UA), while the fatty acids with the highest degree of unsaturation, especially C28:8n3, remain in the non-urea adduct (NUA) fraction to a large extent. Such urea fractionation is conducted under conditions typically employed for the relevant starting material, which conditions are well known or can be readily determined by one of skill in the art. Urea is typically added in amounts (ranging from 0.3 to 5 parts by weight per part of weight of oil) under reaction conditions (for example at temperature between ambient and 80° C.) for periods of time typically employed in the concentrate of commercial concentrated PUFA compositions.

### Filtration:

Some of the oil from distillation 6 (Column 6 Table 3) was cooled down to −15° C. and filtered. The composition of the starting oil, filtrate and filter cake are shown in columns 2, 3 and 4 respectively of Table 5.

The filter cake (column 4, Table 5) was heated to 10° C. and filtered again, the composition of the filtrate and filter cake are shown in columns 5 and 6 respectively of Table 5.

TABLE 5

| Column 1 | 2 | 3 | 4 | 5 | 6 |
|---|---|---|---|---|---|
| | Start | Filtrate | Filter | Filtrate | Filter |
| | | −15° C. | cake | 10° C. | cake |
| | | | −15° C. | | 10° C. |
| Fatty acid | | | | | |
| C20:5n3 | 1.11 | 0.14 | 0.06 | 0.1 | 0.07 |
| C22:0 | | | 2.3 | 6.31 | 1.05 |
| C22:1 | 5.07 | 7.25 | 3.15 | 1.61 | 3.63 |
| C21:5 n3 | 0.96 | 1.17 | 0.75 | | 0.83 |
| C22:5 n6 | 0.19 | 0.98 | 0.94 | 0.68 | 1.27 |
| C22:5 n3 | 2.1 | 3.01 | 1.29 | 0.65 | 1.47 |
| C22:6 n3 | 8.06 | 11.53 | 7.97 | | 0.67 |
| C24:0 | 3.73 | | 4.79 | 34.14 | 5.66 |
| C24:1 | 29.24 | 22.05 | 35.61 | 20.18 | 40.01 |
| C24:1 | 2.25 | 1.18 | 3.2 | 2.05 | 3.57 |
| C25:0 | 0.24 | | 0.51 | 2.26 | 0.04 |
| C24:2 | 0.43 | 0.63 | 0.57 | 0.35 | 0.42 |
| C24:3 | 0.53 | 0.7 | 0.38 | | 0.44 |
| C24:4n3 | 0.68 | 0.95 | 0.45 | 0.26 | 0.48 |
| C24:5n3 | 4.58 | 6.56 | 2.8 | 1.4 | 3.19 |
| C26:0 | 0.42 | | 0.86 | 3.69 | |
| C24:6n3 | 2.22 | 2.9 | 1.43 | 0.67 | 1.63 |
| C26:1 | 6.82 | 1.98 | 11.97 | 6.57 | 13.52 |
| C26:3 | | 0.65 | 0.57 | 0.29 | 0.66 |
| C26:4n3 | 1.43 | 2.03 | 0.91 | 0.45 | 1.03 |
| C26:5n3 | 2.22 | 3.11 | 1.38 | 0.7 | 1.54 |
| C26:6n3 | 4.2 | 6.03 | 2.6 | 1.3 | 2.93 |
| C26:7n3 | 0.48 | 0.72 | 0.3 | 0.15 | 0.34 |
| C28:0 | 0.18 | 0.28 | 0.11 | | 0.12 |
| C28:1 | | 0.17 | 0.1 | | 0.1 |
| C28:2 | 0.32 | 0.43 | 0.23 | 0.11 | 0.25 |
| C28:4n3 | 0.55 | 0.75 | 0.35 | 0.17 | 0.41 |
| C28:5n3 | 0.53 | 0.78 | 0.33 | 0.12 | 0.35 |
| C28:6n3 | 1.2 | 1.75 | 0.74 | 0.36 | 0.84 |
| C28:7n3 | 0.23 | 0.33 | 0.14 | 0 | 0.15 |
| C28:8n3 | 9.39 | 13.57 | 5.8 | 2.86 | 6.47 |
| C30:6n3 | 0.16 | 0.28 | 0.13 | | 0.15 |
| C30:8n3 | 0.22 | 0.39 | 0.14 | | 0.16 |
| C32:5n3 | 0.22 | | | | |
| C32:8n3 | 0.3 | 0.42 | 0.16 | | 0.16 |
| Sum VLC PUFA | 29.89 | 42.98 | 19.41 | 9.19 | 21.60 |
| Sum VLC MUFA | 38.31 | 25.38 | 50.88 | 28.80 | 57.20 |

### Example 2: Cholesterol Removal

Mackerel oil, with the composition described in column 2 of Table 6 below, was reacted to form ethyl esters according to the art.

The content of the ethyl esters of short chain fatty acids of the mackerel oil described in column 2 of Table 6 was reduced by short path distillation (VTA, model VK83-6-SKR-G with degasser). The distillation took place at temperature of 157° C., a flow of 7.4 kg/h and a vacuum of 0.01 mbar. This procedure gave a distillate of 96.5% and a residue of 3.5%. The composition of the ethyl esters of the residue is given in column 3 of Table 6 below. As is well known by the skilled person, the distillate fraction from this step can be utilized for the manufacture of other desired products.

The residue from such distillation (column 3, Table 6) was ethylated as described in the art by sodium ethylate in anhydrous ethanol to reduce the content of glycerides.

The ethylated oil, was then taken through a double distillation in a short path distillation still at a flow of 4.5 ml/min and a pressure of 10⁻³ mbar (VTA, model VKL-70-4-SKR-T) at a temperature of 180° C. The residue (˜10%) was collected as waste while the distillate (˜90%) (column 4, Table 6) was taken further.

### Enzymatic Treatment:

446 g of the distillate (column 4, Table 6) was added 25 g of Lipozyme 435 (Novozyme), and stirred at 80° C. and vacuum (10 mbar) for 36 hours. Similar to what is described in Example 1, the free cholesterol was substantially converted to cholesterol esters in this step.

The oil after enzyme treatment (362 g) was distilled using a short path distillation still (VTA, model VKL-70-4-SKR-T) at a temperature of 180° C., a flow of 4.5 ml/min and a pressure of 10⁻³ mbar, a residue (110 g) containing most of the cholesterol esters, and a distillate (254 g) (column 5, Table 6) were collected. 90 g of the distillate (column 5, Table 6) was distilled using a short path distillation still (VTA, model VKL-70-4-SKR-T) at a temperature of 110° C., a flow of 3.5 ml/min and a pressure of 10⁻³ mbar. A residue of ˜18 g (column 7, Table 6) and a distillate of ˜72 g (column 6, Table 6) were collected.

90 g of the distillate from above (column 5, Table 6) was distilled using a short path distillation still (VTA, model VKL-70-4-SKR-T) at a temperature of 100° C., a flow of 3.5 ml/min and a pressure of 10⁻³ mbar. A residue of ˜45 g (column 9, Table 6) and a distillate of ˜45 g (column 8, Table 6) were collected.

### Urea Fractionation and Filtration:

Some of the oil after enzymatic treatment and distillation, the distillate (Column 5, Table 6), was taken through a urea precipitation procedure to separate fatty acids having the same lengths but different degree of unsaturation.

100 g of urea was mixed with 210 g of ethanol (96%) in a jacketed reactor and heated to 80° C. under stirring. 56 g of oil (column 5, Table 6) was added, and the mixture stirred for 30 min. After cooling to 25° C. the mixture was filtered, the ethanol evaporated, and the mixture filtered a second time. The oil was then washed with 5% citric acid in water and twice with water and dried under vacuum to yield 19.5 g of oil (column 10, Table 6).

TABLE 6

| Column 1 | 2 | 3 | 4 | 5 | 6 | 7 | 8 | 9 | 10 |
|---|---|---|---|---|---|---|---|---|---|
| | Start | R157 | D180 | Enz + | DD110 | DR110 | DD100 | DR100 | Urea |
| | | | | D180 | | | | | prod |
| Fatty acid | | | | | | | | | |
| C14:0 | 6.02 | 2.59 | 2.66 | 2.82 | 4.05 | 0.77 | 4.66 | 0.32 | 0.21 |
| C16:0 | 11.54 | 5.42 | 5.84 | 6.27 | 9.65 | 0.96 | 10.78 | 0.26 | 0.18 |
| C16:1 | 3.79 | 1.82 | 2.03 | 2.16 | 3.63 | 0.35 | 3.74 | 0.09 | 3.09 |
| C18:0 | 1.91 | 1.19 | 1.23 | 1.29 | 1.78 | | 2.19 | 0.07 | |
| C18:1 n9 | 9.86 | 5.9 | 6 | 6.22 | 7.82 | 0.58 | 10.42 | 0.22 | 4.89 |
| C18:1 n7 | 1.84 | 1.09 | 1.13 | 1.2 | 1.58 | 0 | 2.02 | 0.05 | 0.91 |
| C18:3 n3 | 1.24 | 0.76 | 0.75 | 0.76 | 0.9 | 0.06 | 1.29 | 0.03 | 1.81 |
| C18:4 n3 | 4.05 | 2.39 | 1.91 | 2.45 | 2.18 | 0.23 | 4.1 | 0.09 | 6.34 |
| C20:1 | 9.23 | 7.11 | 6.89 | 6.84 | 7.61 | 1.11 | 10.03 | 2.82 | 1.39 |
| C20:4 n6 | 0.51 | 0.35 | 0.36 | 0.37 | 0.43 | 0.05 | 0.53 | 0.12 | 0.8 |
| C20:4 n3 | 1.04 | 0.8 | 0.74 | 0.75 | 0.85 | | | 0 | 1.94 |
| C20:5 n3 | 7.44 | 5.46 | 5.52 | 5.52 | 6.78 | 0.7 | 8.46 | 1.11 | 14.92 |
| C22:1 | 15.66 | 22.05 | 22.04 | 21.97 | 20.52 | 19.17 | 14.12 | 31.88 | 0.75 |
| C21:5 n3 | 0.98 | 1.43 | 1.34 | 1.33 | 1.41 | 1.37 | 0.9 | 1.7 | 0.05 |
| C22:5 n6 | 0.06 | 0.11 | 0.15 | 0.06 | 0.05 | 0.28 | 0.03 | 0.19 | 0.17 |
| C22:5 n3 | 1.36 | 1.59 | 1.56 | 1.58 | 1.61 | 0.85 | 1.34 | 1.79 | 4.04 |
| C22:6 n3 | 11.41 | 12.72 | 12.87 | 13.29 | 13.52 | 8.4 | 11.58 | 14.62 | 31.73 |
| C24:0 | | 0.16 | 0.15 | 0.16 | 0.07 | 0.51 | 0.03 | 0.33 | 0 |
| C24:1 | 1.16 | 7.26 | 7.36 | 7.24 | 3.56 | 20.65 | 1.46 | 15.83 | 0 |
| | | | 0.22 | 0.21 | 0.11 | 0.6 | 0.04 | 0.47 | 0 |
| C24:2 | | | | | | 0.13 | | 0.06 | |
| | | | 0.04 | 0.04 | 0.02 | | | | |
| C24:4 n3 | 0.03 | | 0.09 | 0.04 | 0.05 | 0.17 | 0.03 | 0.16 | 0.1 |
| C24:5 n3 | 0.4 | 1.75 | 1.79 | 1.8 | 1.17 | 3.86 | 0.55 | 3.61 | 4.38 |
| C24:6 n3 | 0.15 | 1.15 | 1.11 | 1.09 | 0.41 | 2.85 | 0.2 | 2.33 | 1.43 |
| C26:0 | | | | 0.55 | | 2.77 | | 1.44 | 0 |
| C26:1 | 0.02 | 1.06 | 1.02 | 0.98 | 0.09 | 4.41 | 0.04 | 2.59 | 0 |
| C26:2 | | | | | | 1.12 | | 0.63 | 0.03 |
| C26:4n3 | 0.03 | 0.51 | 0.5 | 0.47 | 0.1 | 1.99 | 0.04 | 1.19 | 0.44 |
| C26:5n3 | 0.02 | 0.17 | 0.18 | 0.17 | 0 | 0.67 | | 0.41 | 0.35 |
| C26:6n3 | 0.03 | | 0.22 | 0.22 | 0.07 | 0.82 | 0.03 | 0.53 | 0.56 |
| C26:7n3 | | | 0.07 | 0.06 | 0.02 | 0.21 | 0.01 | 0.15 | 0.16 |
| C28:0 | 0.01 | | 0.02 | 0.02 | 0.01 | 0.12 | | 0.07 | 0 |
| C28:1 | 0.01 | | | 0.04 | | 0.17 | | 0.1 | 0.1 |
| C28:2 | 0.01 | 0.23 | 0.19 | 0.18 | 0.01 | 0.93 | 0.01 | 0.49 | 0.03 |
| C28:4n3 | 0.02 | 0.24 | 0.24 | 0.22 | 0.02 | 1.13 | | 0.6 | 0.35 |
| C28:5n3 | | 0.03 | 0.04 | 0.04 | 0.01 | 0.21 | | 0.11 | 0.11 |
| C28:6n3 | 0.03 | 0.43 | 0.53 | 0.49 | 0.05 | 2.48 | 0.01 | 1.34 | 1.24 |
| C28:7n3 | | 0 | 0.02 | 0.02 | | 0.06 | | 0.04 | 0.03 |
| C28:8n3 | 0.08 | 1.53 | 1.4 | 2.38 | 0.18 | 6.73 | 0.08 | 3.66 | 3.73 |
| C30:6 n3 | 0.02 | 0.59 | 0.38 | 0.33 | 0 | 1.9 | | 0.93 | 0.21 |
| C30:8 n3 | 0.05 | | 0.87 | 0.75 | 0 | 4.46 | | 2.19 | 1.63 |
| C32 | 0.01 | 1.29 | | | | | | 0.02 | |
| Sum VLCn | 0.87 | 7.69 | 7.44 | 8.08 | 2.08 | 27.54 | 0.95 | 17.27 | 14.72 |
| PUFA N3 | | | | | | | | | |
| Sum | 1.19 | 8.32 | 8.38 | 8.26 | 3.65 | 25.23 | 1.5 | 18.52 | 0.1 |
| VLCMUFA | | | | | | | | | |

### Separation by Chromatography:

100 mg of the oil from column 7, Table 6 was methylated by standard methods and dissolved in hexane. The hexane phase was eluted through a solid phase extraction (SPE) cartridge with silica coated with AgNO3 (Supelco Discovery™ AG-ION 750 mg/6 ml). After application of the methylated oil in hexane the cartridge was eluted with acetone, yielding an oil with the composition given in column 2, Table 7) and then with 40% acetonitrile (ACN) in acetone, yielding an oil with the composition given in column 3, Table 7.

The above results show that VLCPUFAs and VLCMUFAs can be separated from each other by use of (but not limited to) chromatography, producing compositions with a high or low VLC-PUFA/VLC-MUFA ratio.

TABLE 7

| Column 1 | 2 | 3 |
|---|---|---|
| | Acetone | 40% ACN |
| | | in acetone |
| Fatty acids | | |
| C14:0 | 1.24 | |
| C16:0 | 1.38 | |
| C16:1 | 0.49 | |
| C18:0 | 0.2 | |
| C18:1 n9 | 0.76 | |
| C18:1 n7 | 0.17 | |
| C20:1 | 1.56 | |
| C20:4 n6 | | |
| C20:4 n3 | | |
| C20:5n3 | | 1.43 |
| C22:1 | 29.8 | |
| C22:0 | 2.18 | |
| C21:5n3 | | 0.34 |
| C22:5n6 | | 2.06 |
| C22:5n3 | | 13 |
| C22:6n3 | 0 | 13.01 |
| C24:0 | 2.25 | 0.01 |
| C24:1 | 31.6 | 0.17 |
| | 0.79 | |
| C24:2 | | 0.27 |
| C24:4n3 | | 0.08 |
| C24:5n3 | 0.17 | 9.56 |
| | 0.38 | |
| | 2.6 | |
| C24:6n3 | | 2.59 |
| C26:0 | 4.8 | |
| C26:1 | 7.12 | |
| C26:2 | 0.59 | 0.18 |
| C26:4n3 | | 5.34 |
| C26:5n3 | | 1.7 |
| C26:6n3 | | 2.16 |
| C26:7n3 | | 0.49 |
| C28:0 | 0.05 | 0.45 |
| C28:1 | 0 | 0.46 |
| C28:2 | | 2.82 |
| C28:4n3 | 0.02 | 3.34 |
| C28:5n3 | 0.01 | 0.5 |
| C28:6n3 | | 7.06 |
| C28:7n3 | | 0.22 |
| C28:8n3 | | 18.15 |
| C30:5n3 | | |
| C30:6n3 | | 7.24 |
| C30:8n3 | | 15.61 |
| Sum VLC PUFA n3 | 3.77 | 74.49 |
| Sum VLCMUFA | 38.72 | 0.46 |
| Ratio VLC-PUFA/ | 0.095 | 161.9 |
| VLC-MUFA | | |

### Example 3: VLCUSFA Compositions Obtained without Enzyme Treatment

A residue (column 2, Table 8), from a commercial scale distillation of an ethylated sardine and mackerel oil utilized to produce an omega-3-acid concentrate containing about 36% EPA and about 25% DHA, was distilled using a short path distillation still (VTA, model VKL-70-4-SKR-T) at a temperature of 180° C., a flow of 5 ml/min and a pressure of 10⁻³ mbar. A residue (column 4, Table 8) and a distillate (column 3, Table 8) was collected. The same starting residue (column 2) was also converted to ethyl esters according to the art, the analytical results are given in column 5, Table 8.

The distillate in column 3, Table 8, was further distilled using a short path distillation still (VTA, model VKL-70-4-SKR-T) at a temperature of 115° C., a flow of 5 ml/min and a pressure of 10⁻³ mbar, a residue (column 7, Table 8) and a distillate (column 6, Table 8) were collected.

The distillate from the distillation at 180° C. (column 3) was much lower in total cholesterol compared to the starting oil (8.67 vs 37.37 mg/g); this positive effect is caused by the cholesterol esters being collected in the residue fraction (column 4). The distillate contains mainly cholesterol in the form of free cholesterol.

As can be seen from columns 2 and 5, an ethylation of the (same) starting oil does not change the content of total cholesterol, but some cholesterol ester is converted to free cholesterol. As free cholesterol is more difficult to separate from VLCMUFA and VLC-PUFA by distillation, it has been found advantageous to remove as much as possible of the cholesterol as cholesterol ester in a residue fraction during distillation. This is illustrated by comparison of total cholesterol of oil in column 2 vs column 5 in Table 8.

Further distillation of the distillate in column 3, Table 8, gave a residue (column 7) that was enriched in VLCPUFA (6.59%) and VLCMUFA (9.4%), the cholesterol (total) was slightly increased to 11.9 mg/g from 8.7 (column 3), but efficiently reduced from the start concentration of 37.4 mg/g (column 2).

TABLE 8

| Column 1 | 2 | 3 | 4 | 5 | 6 | 7 |
|---|---|---|---|---|---|---|
| | Residue | Dest | Residue | Ethylated | D115 | R115 |
| | 1 | 180 | 180 | residue 1 | | |
| EE (%) | 56.45 | 72.38 | 9.6 | 80.92 | 93.73 | 53.71 |
| MG (%) | 6.17 | 0 | 30.78 | 0.13 | 0 | 1.26 |
| DG (%) | 16.48 | 3.19 | 57.39 | 4.92 | 0 | 6.68 |
| TG (%) | 20.91 | 24.43 | 0.14 | 14.03 | 6.27 | 38.35 |
| Total | 37.4 | 8.7 | 89.5 | 39.7 | 0.5 | 11.9 |
| cholesterol | | | | | | |
| (mg/g) | | | | | | |
| Free | 6.3 | 8.3 | 0.3 | 19.4 | 0.5 | 11.6 |
| Cholesterol | | | | | | |
| (mg/g) | | | | | | |
| C14:0 | 1.46 | 1.35 | 6.09 | 1.46 | 2.69 | 1.4 |
| C16:0 | 5.25 | 4.25 | 17.64 | 5.39 | 6.54 | 5.68 |
| C16:1 | 1.76 | 1.62 | 6.84 | 1.99 | 2.71 | 1.9 |
| C16:4 | 0.18 | 0.04 | 1.11 | 0.32 | 0.41 | 0.05 |
| C18:0 | 1.48 | 1.34 | 3.9 | 1.78 | 1.39 | 1.94 |
| C18:1 n9 | 2.49 | 2.26 | 8.16 | 2.87 | 2.96 | 3.07 |
| C18:1 n7 | 1.17 | 0.81 | 2.76 | 1.08 | 0.91 | 1.07 |
| C18:2 n6 | 0.28 | 0.34 | 1.29 | 0.28 | 0.50 | 0.44 |
| C18:3 n3 | 0.12 | 0.21 | 0.05 | 0.02 | 0.24 | 0.27 |
| C18:4 n3 | 0.07 | 0.63 | 2.14 | 0.77 | 0.76 | 0.82 |
| C20:1 | 1.47 | 1.5 | 1.33 | 1.52 | 2.12 | 1.27 |
| C20:4 n6 | 0.89 | 0.95 | 0.11 | 1.07 | 1.35 | 0.59 |
| C20:4 n3 | 0.72 | 0.85 | 0.73 | 0.84 | 1.16 | 0.5 |
| C20:5 n3 | 14.39 | 13.22 | 14.81 | 14.17 | 19.09 | 7.81 |
| C22:0 | 0.51 | 0.74 | 0.18 | 0.7 | 0.45 | 0.86 |
| C22:1 | 6.28 | 6.97 | 2.1 | 6.38 | 5.40 | 8.02 |
| C21:5 n3 | 1.01 | 1 | 0.29 | 0.97 | 0.78 | 1.21 |
| C22:5 n6 | 1.01 | 0.14 | 0.4 | 0.46 | 0.36 | 0.82 |
| C22:5 n3 | 6.78 | 7.17 | 2.02 | 6.66 | 6.44 | 6.6 |
| C22:6 n3 | 32.63 | 33.56 | 11.85 | 31.6 | 32.04 | 28.67 |
| C24:0 | 0.38 | 0.3 | 0.1 | 0.05 | 0.87 | |
| C24:1 | 4.78 | 4.83 | 0.71 | 4.38 | 1.04 | 7.41 |
| C24:1 | 0.5 | 0.53 | 0.08 | 0.51 | 0.13 | 0.66 |
| C24:2 | | 0.04 | | 0.03 | 0.05 | 0.05 |
| | 0.02 | 0.09 | | | | |
| C24:4n3 | 0.01 | 0.09 | | 0.07 | 0.01 | 0.13 |
| C24:5n3 | 0.82 | 0.95 | 0.14 | 0.85 | 0.28 | 1.36 |
| C26:0 | 0.03 | 0.05 | 0.06 | 0.04 | 0.03 | 0.08 |
| C24:6n3 | 0.35 | 0.4 | 0.06 | 0.36 | 0.14 | 0.56 |
| C26:1 | 0.66 | 0.73 | 0.34 | 0.65 | 0.04 | 1.25 |
| C26:3 | 0.07 | 0.08 | | 0.07 | | 0.14 |
| C26:4n3 | 0.16 | 0.17 | | 0.16 | 0.01 | 0.29 |
| C26:5n3 | 0.26 | 0.26 | 0.06 | 0.24 | 0.03 | 0.44 |
| C26:6n3 | 0.45 | 0.49 | 0.06 | 0.44 | 0.08 | 0.81 |
| C26:7n3 | 0.06 | 0.06 | | 0.05 | 0.01 | 0.1 |
| C28:0 | 0.06 | 0.06 | 0.01 | 0.05 | 0.05 | 0.08 |
| C28:1 | 0.38 | 0.01 | 0.01 | | | 0.01 |
| C28:2 | 0.05 | 0.05 | 0.01 | 0.05 | | 0.08 |
| C28:4n3 | 0.08 | 0.07 | 0.07 | 0.08 | | 0.11 |
| C28:5n3 | 0.04 | 0.04 | 0.01 | 0.04 | | 0.05 |
| C28:6n3 | 0.14 | 0.16 | 0.01 | 0.06 | | 0.26 |
| C28:7n3 | 0.01 | 0.03 | | | | 0.05 |
| C28:8n3 | 1.01 | 1.11 | 0.12 | 0.99 | 0.06 | 1.86 |
| C30:6n3 | 0.06 | 0.07 | | | | 0.13 |
| C30:8n3 | 0.07 | 0.06 | 0.06 | | | 0.11 |
| | | | | | | 0.1 |
| C32 | 0.05 | 0.07 | 0.13 | 0.06 | | 0.11 |
| Sum VLC | 3.69 | 4.25 | 0.78 | 3.51 | 0.64 | 6.59 |
| PUFA n3 | | | | | | |
| VLCMUFA | 6 | 6.15 | 1.14 | 5.59 | 1.25 | 9.4 |

The example shows how cholesterol (total and free) content changes during use of distillation for purification and up-concentration of oils rich in VLC-PUFAs and VLC-MUFAs. It has hence been found that cholesterol in the form of cholesterol esters can be separated from the VLC-PUFA and VLC-MUFA compositions by distillation, while the free cholesterol is difficult to separate from the VLC-PUFAs and VLC-MUFAs by distillation only.

### Example 4: Separation of Cholesterol by Distillation

A residue (column 2, Table 9), from a commercial scale distillation of an ethylated sardine and mackerel oil utilized to produce an omega-3-acid concentrate containing about 36% EPA and about 25% DHA, was distilled using a short path distillation still (VTA, model VKL-70-4-SKR-T) at a temperature of 180° C., a flow of 5 ml/min and a pressure of 10⁻³ mbar. A distillate of ˜90% (column 3, Table 9) and a residue of about 10% were collected. The distillate was ethylated as described in the art, the product was analyzed as shown in column 4, Table 9. The ethylated oil was finally distilled using a short path distillation still (VTA, model VKL-70-4-SKR-T) at a temperature of 110° C., a flow of 5 ml/min and a pressure of 10⁻³ mbar. A residue of about 60% (column 5, Table 9) was collected and analyzed.

The analytical results show that during the first distillation at 180° C., the total cholesterol in the distillate was reduced (Column 3, Table 9), mainly because the cholesterol ester was removed in the residue fraction. In the ethylation step the total cholesterol stayed basically the same, while the fatty acids were converted to ethyl ester (EE) from triglycerides (TG). The final distillation step increased the concentration of very long chain fatty acids in the residue, but the cholesterol concentration also increased.

The example illustrates how the VLC-PUFA and VLCMUFA content and the cholesterol content (free and total) change through purification and up-concentration. By the process that is described in this example the content of cholesterol esters is reduced effectively, while free cholesterol increases as the VLC-PUFA and VLC-MUFA are up-concentrated.

TABLE 9

| Column 1 | 2 | 3 | 4 | 5 |
|---|---|---|---|---|
| | Residue | D180 | Ethylated | R110 |
| EE (%) | 56.45 | 73.23 | 81.78 | 66.15 |
| TG (%) | 6.17 | 0 | 0 | 0 |
| DG (%) | 16.48 | 1.35 | 0.58 | 2.49 |
| MG (%) | 20.91 | 25.42 | 17.64 | 31.36 |
| olig (%) | 0 | 0 | 0 | 0 |
| Total Cholesterol | 37.37 | 7.13 | Not | 17.2 |
| (mg/g) | | | analyzed | |
| F Cholesterol (mg/g) | 6.32 | 8.9 | 9.1 | 18 |
| Fatty acid | | | | |
| C14:0 | 1.46 | 0.67 | 0.7 | 0.06 |
| C16:0 | 5.25 | 2.93 | 2.97 | 0.22 |
| C16:1 | 1.76 | 1.03 | 1.04 | 0.07 |
| C16:4 | 0.18 | 0.17 | 0.17 | 0.01 |
| C18:0 | 1.48 | 1.26 | 1.26 | 0.2 |
| C18:1 n9 | 2.49 | 1.81 | 1.83 | 0.23 |
| C18:1 n7 | 1.17 | 0.69 | 0.7 | 0.09 |
| C18:2 n6 | 0.28 | 0.26 | 0.26 | 0.03 |
| C18:3 n3 | 0.12 | 0.17 | 0.17 | 0.02 |
| C18:4 n3 | 0.07 | 0.5 | 0.51 | 0.07 |
| C20:1 | 1.47 | 1.55 | 1.57 | 0.81 |
| C20:4 n6 | 0.89 | 0.97 | 0.98 | 0.35 |
| C20:4 n3 | 0.72 | 0.88 | 0.89 | 0.43 |
| C20:5 n3 | 14.39 | 13.58 | 13.67 | 5.03 |
| C22:0 | 0.51 | 0.79 | 0.77 | 1.14 |
| C22:1 | 6.28 | 7.38 | 7.41 | 9.78 |
| C21:5n3 | 1.01 | 1.05 | 1.07 | 1.54 |
| C22:5 n6 | 1.01 | 0.58 | 0.6 | 0.24 |
| C22:5 n3 | 6.78 | 7.62 | 7.68 | 9.04 |
| C22:6 n3 | 32.63 | 35.48 | 35.96 | 40.46 |
| C24:0 | 0.38 | 0.07 | 0.005 | 0.08 |
| C24:1 | 4.78 | 5.09 | 5.07 | 9.72 |
| C24:1 | 0.5 | 0.54 | 0.5 | 0.91 |
| C24:2 | | 0.02 | 0.03 | 0.07 |
| | 0.02 | 0.04 | 0.02 | 0.16 |
| C24:4n3 | 0.01 | 0.07 | 0.09 | 0.21 |
| C24:5n3 | 0.82 | 1.03 | 1.03 | 1.87 |
| C26:0 | 0.03 | 0.06 | 0.06 | 0.11 |
| C24:6n3 | 0.35 | 0.42 | 0.42 | 0.77 |
| C26:1 | 0.66 | 0.7 | 0.69 | 1.39 |
| C26:3 | 0.07 | 0.08 | 0.08 | 0.15 |
| C26:4n3 | 0.16 | 0.17 | 0.17 | 0.34 |
| C26:5n3 | 0.26 | 0.27 | 0.27 | 0.53 |
| C26:6n3 | 0.45 | 0.5 | 0.5 | 1 |
| C26:7n3 | 0.06 | 0.06 | 0.07 | 0.13 |
| C28:0 | 0.06 | 0.06 | 0.06 | 0.02 |
| C28:1 | 0.38 | 0.02 | 0.02 | 0.02 |
| C28:2 | 0.05 | 0.04 | 0.04 | 0.08 |
| C28:4n3 | 0.08 | 0.08 | 0.07 | 0.16 |
| C28:5n3 | 0.04 | 0.04 | 0.05 | 0.1 |
| C28:6n3 | 0.14 | 0.14 | 0.14 | 0.27 |
| C28:7n3 | 0.01 | 0.03 | 0.03 | 0.05 |
| C28:8n3 | 1.01 | 1.02 | 1.01 | 1.11 |
| C30:6n3 | 0.06 | 0.02 | 0.04 | 0.08 |
| C30:8n3 | 0.07 | 0.06 | 0.04 | 0.09 |
| C32 | 0.05 | 0.05 | 0.05 | 0.09 |
| Sum VLC PUFA n3 | 3.69 | 4.14 | 4.14 | 7.22 |
| VLCMUFA | 6 | 6.39 | 6.32 | 12.04 |

### Example 5: VLCUSFA Compositions with Very Low Cholesterol Content

A crude “1812” oil (an acronym for an oil intended to result in a commercial product containing about 18% EPA (C20:5n3) and 12% DHA (C22:6n3)) from a mixture of sardine and mackerel oil (column 2, Table 10), was added 7% of a C14-C18 fatty acid ethyl ester fraction obtained as a by-product from the manufacture of commercial fatty acid concentrates, and then distilled using a short path distillation still (VTA, model VKL-70-4-SKR-T) at a temperature of 180° C., a flow of 5 ml/min and a pressure of 10-3 mbar. A residue of 90% (column 3, Table 10) containing slightly less than half of the total cholesterol that was present in the starting crude oil, and a distillate of 10% was collected. The residue (column 3, Table 10) was ethylated as described in the art, e.g. by reacting the oil with ethanol, the product was analysed and this is shown in column 4, Table 10. The ethylated oil was then treated with enzyme (lipase) at 80° C. under vacuum overnight, similar to what is described in Example 1. The resulting oil is shown in column 5, Table 10. The enzyme treated oil was finally distilled using a short path distillation still (VTA, model VKL-70-4-SKR-T) at a temperature of 180° C., a flow of 5 ml/min and a pressure of 10-3 mbar. A residue of about 10% and a distillate (column 6, Table 10) of about 90% was collected and analysed.

The analytical results show that during the first distillation at 180° C., the total cholesterol was reduced, mainly because free cholesterol was removed with the distillate fraction. In the ethylation the content of total cholesterol remained unchanged, while the fatty acids were converted to EE from TG. The following enzyme treatment converted the remaining free cholesterol to cholesterol esters. During the final distillation step the cholesterol esters and glycerides remained in the residue, while the distillate was very low in total cholesterol. A distillate with total cholesterol below 1 mg/g was produced. The fatty acid profile remained unchanged during the process steps, with contents of around 0.5% of each of VLCPUFAs and VLCMUFAs. The skilled person will realise that a substantial reduction of cholesterol content of the “1812” oil described above could also be obtained without carrying out a first step of addition of a C14-C18 fatty acid ethyl ester fraction, followed by distillation using a short path distillation still.

Further, the skilled person will realise that the method for reducing cholesterol content as described above could, for the ethylation step, be performed not only via a preferred embodiment of reacting the starting oil with ethanol in order to form ethyl esters, but also from reacting the oil with other alcohols, e.g. like methanol and propanol, to form the corresponding esters.

WO2004/007655 describes on page 18, line 13—page 19, line 9, a process to reduce the content of total cholesterol of C1-04 (methyl-butyl) esters from fish oils, but does not include examples or claims to support the description. WO2004/007655 solely refers to the long chain C20-C22 PUFAs such as EPA and DHA (see for example page 2, line 16—page 3, line 13, and page 13, lines 5-24), and is completely silent as to the presence of VLCFAs in marine oils. As VLCFAs distil at higher temperatures than LCFAs, it is very surprising that the VLCFA esters can be substantially separated from the cholesterol esters by a procedure such as short path distillation/molecular distillation. Compared to the process of WO2004/007655, an important benefit of the current process is that an esterification of the remaining free cholesterol of the residue is performed, providing a more complete removal of cholesterol.

TABLE 10

| Column 1 | 2 | 3 | 4 | 5 | 6 |
|---|---|---|---|---|---|
| | Start | R180 | Ethylated | Enzyme | D180 |
| Tot Chol | 5.54 | 2.68 | 2.36 | 2.94 | 0.37 |
| (mg/g) | | | | | |
| F Chol | 3.94 | 0.16 | 0.25 | 0.01 | 0.34 |
| (mg/g) | | | | | |
| Fatty acid | | | | | |
| C20:1 | 1.25 | | | | 1.26 |
| C20:4 n6 | | | | | 1.11 |
| C20:4 n3 | | | | | 0.78 |
| C20:5 n3 | 16.64 | | | | 16.55 |
| C22:0 | 0.16 | | | | 0.18 |
| C22:1 | 1.39 | | | | 1.42 |
| C21:5 n3 | 0.11 | | | | 0.26 |
| C22:5 n3 | 1.83 | | | | 1.82 |
| C22:6 n3 | 11.33 | | | | 11.12 |
| C24:0 | 0.09 | | | | 0.06 |
| C24:1 | 0.43 | | | | 0.45 |
| C24:1 | 0.03 | | | | |
| C24:2 | | | | | |
| C24:4n3 | 0.09 | | | | |
| C24:5n3 | 0.15 | | | | 0.16 |
| C26:0 | | | | | |
| C24:6 | 0.11 | | | | 0.12 |
| C26:1 | 0.03 | | | | 0.04 |
| C26:3n3 | 0.02 | | | | |
| C26:4n3 | 0.23 | | | | 0.01 |
| C26:5n3 | 0.05 | | | | 0.04 |
| C26:6n3 | 0.01 | | | | 0.04 |
| C26:7n3 | 0.01 | | | | 0.01 |
| C28:0 | | | | | |
| C28:1 | 0.01 | | | | |
| C28:4n3 | 0.03 | | | | |
| C28:5n3 | 0.01 | | | | 0.01 |
| C28:6n3 | | | | | |
| C28:7n3 | | | | | |
| C28:8n3 | 0.1 | | | | 0.15 |
| Sum VLC | 0.81 | 0 | 0 | 0 | 0.54 |
| PUFA n3 | | | | | |
| VLCMUFA | 0.49 | 0 | 0 | 0 | 0.49 |

### Example 6: Colour Assessments

Two different residues (columns 2 and 3, Table 11) from commercial scale distillations of ethylated sardine and mackerel oil utilized to produce an omega-3-acid concentrate were analysed for colour Gardner, and compared with the following two purified and up/concentrated VLCPUFA/VLCMUFA oils;

The oil of Column 6, Table 3, provided in column 4, Table 11.

The oil of Column 2, Table 19 (Example 9), provided in column 5, Table 11.

TABLE 11

| Column 1 | 2 | 3 | 4 | 5 |
|---|---|---|---|---|
| | Residue | Residue | Purified/ | Purified/ |
| | A | B | upconcentrated | upconcentrated |
| | | | VLCPUFA/ | VLCPUFA/ |
| | | | VLCMUFA | VLCMUFA |
| | | | (Column 6, | (Column 2, |
| | | | Table 3) | Table 19) |
| Colour | 12 | 13.7 | 6.7 | 5 |
| Gardner | | | | |

The results show that the residue fractions are very high in colour, while a purified VLCUSFA composition (final product) obtained by the distillation and other purification/up-concentration steps, such as those disclosed by the present invention, have achieved an acceptable colour.

### Example 7: VLCUSFA Compositions Further Purified with Active Carbon

Historical data on benzo(A)pyrene (BAP) and polyaromatic hydrocarbons (4PAH) from two residues (column 2 and 3, Table 12) from commercial scale distillations of ethylated sardine and mackerel oil utilized to produce omega-3-acid concentrates, were compared to a purified and up/concentrated VLCPUFA & VLCMUFA concentrate (column 4, Table 12, the same oil as in column 6, Table 3) before and after active carbon (AC) treatment (Column 4 and 5, Table 12).

TABLE 12

| Column 1 | 2 | 3 | 4 | 5 |
|---|---|---|---|---|
| | Residue 2 | Residue 3 | Purified/ | Purified/ |
| | | | upconcentrated | upconcentrated/ |
| | | | | AC treated |
| BAP | 2.80 μg/kg | 4.20 μg/kg | 1.6 μg/kg | <0.5 μg/kg |
| 4PAH* | 11.98 μg/kg | 12.6 μg/kg | 2.4 μg/kg | <0.5 μg/kg |

*4PAH is defined as the sum of benz(a)anthracene, chrysene, benzo(b)fluoranthenes and benzo(a)pyrene

The residue fractions comprise above acceptable/legal values for BAP and 4PAH. The purified compositions however, (column 4, Table 12) comprise lower and below acceptable/legal levels of these pollutants. The active carbon treated oil is very low on both environmental pollutants.

### Example 8: Preparation of High Concentration VLCUSFA Compositions

Some of the oil from the last distillation (Example 1, Table 3, Column 6) was taken through a urea precipitation procedure. Urea fractionation is a method for isolating separate fractions of fatty acids having identical chain lengths but different degrees of unsaturation.

300 g of urea was mixed with 600 g of ethanol (96%) in a jacketed reactor and heated to reflux under stirring. 150 g of the oil (Example 1, Table 3, column 6) was added, and the mixture stirred under reflux for 30 minutes. After cooling to 25° C. the mixture was filtered, the ethanol of the filtrate was partially evaporated, and the mixture filtered a second time. The oil was then washed with 5% citric acid in water and twice with water and dried under vacuum to yield an oil with the fatty acid composition shown in column 2 of Table 13. This product oil was further distilled using a short path distillation still (VTA, model VKL-70-4-SKR-T flow of 3.5 ml/min and a pressure of 10⁻³ mbar). The oil was first distilled at 116°, to remove a light fraction. The residue was then distilled at a temperature of 145° C., and the distillate was collected. The distillate was further distilled at 112° C., the residue from this distillation was taken further and distilled at a temperature of 110° C. The residue fraction (was taken further and) had the fatty acid composition shown in column 3 of Table 13.

TABLE 13

| Column 1 | 2 | 3 | 4 | 5 | 6 |
|---|---|---|---|---|---|
| | Urea | R110 | Hydrol. + | Hydrol. + | Hydrol. + |
| | 200% | | Flash | Flash | Flash |
| Fatty acids | | | Fraction 1 | Fraction 2 | Fraction 3 |
| C20:5 n3 | 0.49 | 0.121 | | 0.16 | 0.37 |
| C22:1 | 4.59 | 0.15 | 0.32 | | 0.11 |
| C21:5n3 | 0.56 | | | | |
| C22:5 n3 | 4.54 | 0.39 | 0.16 | 0.52 | 1.24 |
| C22:6n3 | 13.85 | 0.1 | 0.13 | 0.17 | |
| C24:1 | 4.69 | 1.98 | 3.97 | 0.91 | 0.37 |
| C24:2 | 0.57 | | 0.94 | | |
| C24:3 n3 | 0.93 | 0.48 | 0.44 | | |
| C24:4 n3 | 1.45 | 0.36 | | 0.21 | 0.14 |
| C24:5 n3 | 9.7 | 1.19 | 1.20 | 1.22 | 0.61 |
| C24:6 n3 | 3.4 | 0.30 | 0.18 | 0.38 | 0.47 |
| C26:1 | 0.62 | 0.70 | 1.77 | 0.67 | 0.27 |
| C26:3 | 0.77 | | | | |
| C26:4n3 | 2.64 | 4.60 | 7.52 | 2.46 | 1.01 |
| C26:5 n3 | 4.4 | 5.02 | 6.77 | 3.75 | 3.93 |
| C26:6 n3 | 9.01 | 8.13 | 7.47 | 8.62 | 4.71 |
| C26:7 n3 | 1.11 | 0.66 | 0.39 | 0.81 | 0.77 |
| C28:1 | | | | | |
| C28:4n3 | 1.17 | 2.53 | 1.82 | 0.66 | 0.33 |
| C28:5 n3 | 1.04 | 3.05 | 4.82 | 2.10 | 0.91 |
| C28:6 n3 | 2.32 | 7.58 | 8.85 | 5.43 | 1.65 |
| C28:7 n3 | 0.44 | 2.98 | 2.19 | 1.41 | 1.05 |
| C28:8 n3 | 18.4 | 46.69 | 30.51 | 61.31 | 70.76 |
| C30:6 n3 | 0.2 | 1.98 | 2.99 | 2.57 | 1.78 |
| C30:8 n3 | 0.32 | 1.56 | 2.59 | 1.26 | 0.40 |
| C32 | 0.43 | 1.56 | 0.31 | 0.82 | 0.34 |
| Sum | 58.3 | 88.67 | 78.99 | 93.01 | 88.86 |
| VLCPUFA | | | | | |
| VLCMUFA | 5.31 | 2.68 | 5.74 | 1.58 | 0.64 |

Part of the residue oil (from distillation, i.e. Table 13, column 3) was hydrolysed (Hydrol.), by reacting 7.5 g of the oil with 1.5 g KOH in 30 ml 96% ethanol. After heating at 50° C. for 1 hour the solution was cooled and quenched with 100 ml of water saturated with citric acid. The free fatty acids (FFA) were extracted out with ethyl acetate and washed with water. Evaporation of the organic phase yielded 7 grams of oil.

This oil was taken through a column (2.5 cm diameter) of silica (40 g silica gel 60 0.063-0.300 mm). First the column was eluted with 200 ml of isooctane, no oil was found in this fraction. The column was then eluted with 100 ml of 15% ethyl acetate in isooctane, this fraction (˜1.5 g) (fraction 1) had a composition given in column 4 of Table 13. Elution with another 100 ml of 15% ethyl acetate in isooctane yielded fraction 2 (˜3 g), with the composition given in column 5. Elution with another 100 ml of 15% ethyl acetate in isooctane yielded fraction 3 (˜1.5 g), with the fatty acid composition given in column 6 of Table 13.

Compositions with a high concentration of VLCUSFAs and a high purity are hence obtained. Particularly, the fatty acid C28:8 n3 is obtained in high concentrations.

### Example 9: Preparation of Compositions of VLCUSFAs on Triglyceride Form with Reduced Cholesterol Content

47.82 kg of a residue, from a commercial scale distillation of an ethylated sardine and mackerel oil utilized to produce an omega-3-acid concentrate containing about 36% EPA and about 25% DHA, with the fatty acid composition shown in Column 2 of Table 14, was taken into an ethylation process to reduce the remaining content of glycerides, by reacting the residue with 7 w % (of oil weight) of 2% sodium ethoxide in absolute ethanol. The mixture was stirred under reflux for 1 hour at 80° C. The excess ethanol was then evaporated under vacuum. The stirring was stopped, and after 30 minutes a small dark heavy phase of glycerol was drained out of the reactor via the bottom valve. The oil was then washed with water containing 5% citric acid and twice with water. The oil phase was then dried under vacuum at 40-50° C., the oil with the composition shown in Column 3 of Table 14 was taken directly into the next step.

TABLE 14
The fatty acid profile and cholesterol content of different fractions
during purification and up-concentration of VLCFAs. Results are
given as area percentages (A %); in chromatograms from size
exclusion chromatography (SEC) for ethyl esters (EE),
monoglycerides (MG), diglycerides (DG) and triglyceries (TG),
and as A % from gas chromatography (GC) for the fatty acid
analyses. The skilled person will realize that the fatty acid
composition is not altered by ethylation and enzyme treatment.
For this reason, fatty acid compositions have not been analysed
for column 3 and 4.
Column 2: Residue fraction
Column 3: After ethylation
Column 4: After enzyme treatment as described below
Column 5: Distillate from single distillation of enzymatic
treated oil of column 4 (as described below)
Column 6: Residue from distillations of the oil in column 5
(as described below)

| Column 1 | 2 | 3 | 4 | 5 | 6 |
|---|---|---|---|---|---|
| | Start | Ethylated | Enzyme | Enzym + | Residue |
| | | | treated | D190 | from |
| | | | | | distillation |
| EE (%) | | 91.30 | 83.52 | 93.01 | 70.49 |
| MG (%) | | 7.69 | 5.7 | 6.64 | 28.28 |
| DG (%) | | 1.01 | 4.20 | 0.35 | 1.07 |
| TG (%) | | 0 | 6.58 | 0 | 0.16 |
| Free cholesterol | 13.2 | 12.4 | 0.5 | 0.6 | 2.9 |
| (mg/g) | | | | | |
| Total cholesterol | 14.7 | >12.4 | 12.6 | 0.6 | 2.9 |
| (mg/g) | | | | | |
| Absorbance | 0.30 | 0.30 | 0.65 | | |
| 233 nm | | | | | |
| Fatty acid | | | | | |
| C18:0 | 0.34 | | | | |
| C18:1 n9 | 0.81 | | | | |
| C18:1 n7 | 0.28 | | | 0.61 | |
| C18:2 n6 | | | | | |
| C18:3 n3 | | | | | |
| C18:4 n3 | | | | | |
| C20:1 | 0.98 | | | 0.37 | |
| C20:4 n6 | | | | | |
| C20:4 n3 | | | | 0.33 | |
| C20:5 n3 | 13.83 | | | 13.29 | 0.24 |
| C22:0 | 0.18 | | | 0.63 | 0.82 |
| C22:1 | 1.45 | | | 1.49 | 2.73 |
| C21:5 n3 | | | | | |
| C22:5 n6 | 0.81 | | | 0.82 | 0.82 |
| C22:5 n3 | 8.43 | | | 7.96 | 9.74 |
| C22:6 n3 | 56.42 | | | 54.29 | 49.87 |
| C24:0 | 0.33 | | | 0.36 | 1.55 |
| C24:1 | 2.04 | | | 2.09 | 12.74 |
| C24:2 | | | | | |
| C24:4n3 | 0.15 | | | 0.14 | 0.78 |
| C24:5n3 | 0.26 | | | 0.26 | 1.29 |
| C24:6n3 | 0.29 | | | 0.26 | 1.34 |
| C26:1 | 0.13 | | | 0.13 | 0.95 |
| C26:3n3 | | | | | |
| C26:4n3 | 0.08 | | | 0.06 | 0.50 |
| C26:5n3 | 0.07 | | | 0.10 | 0.51 |
| C26:6n3 | 0.15 | | | 0.16 | 0.99 |
| C26:7n3 | 0.08 | | | 0.05 | 0.39 |
| C28:0 | | | | | |
| C28:1 | | | | | |
| C28:2 | | | | | |
| C28:4n3 | | | | | |
| C28:5n3 | 0.03 | | | 0.03 | 0.12 |
| C28:6n3 | 0.01 | | | 0.01 | 0.23 |
| C28:7n3 | 0.02 | | | 0.02 | 0.18 |
| C28:8n3 | 0.44 | | | 0.45 | 3.07 |
| Sum VLCPUFA | 1.58 | | | 1.54 | 9.4 |
| VLCMUFA | 2.17 | | | 2.22 | 13.69 |

### Enzymatic Treatment:

The resulting oil material from the ethylation step (column 3, Table 14) was added 4.8 kg of immobilised enzyme (Lipozyme 435, Novozymes), and the mixture was stirred at 80° C. and vacuum (10 mbar) for 48 hours. After cooling and filtration, the obtained oil material (42.16 kg) was taken to distillation.

The results from the analysis of samples during enzymatic treatment are shown below in table 15.

TABLE 15

| Column 1 | 2 | 3 | 3 |
|---|---|---|---|
| Time | Start | 24 h | 48 h |
| Free | 12.4 | 2.7 | 0.53 |
| Cholesterol | | | |
| mg/g | | | |
| EE (%) | 91.3 | | 83.52 |
| MG (%) | 7.69 | | 5.7 |
| DG (%) | 1.1 | | 4.20 |
| TG (%) | 0 | | 6.58 |

The results provided in Table 15 show that the free cholesterol is gradually reduced from 12.4 mg/g to 0.53 mg/g over the 48 h reaction time. This means that during the enzymatic step the free cholesterol is converted to cholesterol esters. This surprisingly shows that the lipase enzyme accepts free-cholesterol as the alcohol substrate in the enzymatic synthesis of cholesterol ester. Normally such transformations are done by cholesterol esterase enzymes. The process described above can also be carried out utilizing other relative amounts, and other sources, of suitable enzyme preparations, as well as utilizing other reaction conditions, including other reaction times and vacuums than described in this example, and/or by including additional procedures that can be utilized to bring the reaction to completion, including procedures for removing ethanol that is formed as a by-product during the transesterification reactions.

During the enzymatic reaction, the amount of monoglycerides (MG) was reduced, and amounts of di- and triglycerides (DG, TG) were increased. It should be noted that the SEC method, which is similar to that described in the European Pharmacopoeia and US Pharmacopeia monographs for omega-3-acid triglycerides, omega-3-acid ethyl esters and fish oils, probably overestimates the MG content and underestimate the ethyl ester (EE) content in samples high in long chain fatty acids, as the long chain EEs will have similar molecular size as the shorter chain MGs, and for this reason partly coelute with the MGs. Therefore, the real content of MG in the sample after 48 hours is probably low.

The enzyme treated oil, after 48 hours (column 4, Table 14), was then taken through a single distillation in a short path distillation (VTA, model VK83-6-SKR-G with degasser). The temperature of the first column was 190° C. (flow of 4 kg/h and a vacuum of 0.01 mbar), the residue was collected as waste while the distillate was taken further. Surprisingly, the total cholesterol of the distillate (column 5, Table 14) was only 0.6 mg/g, being reduced from 14.7 mg/g in the starting oil (column 2, Table 14). This very substantial reduction in total cholesterol was caused by the surprising esterification of free cholesterol during the enzymatic treatment, and that the cholesterol esters could be removed in the residue fraction from the first of the distillation steps as described above.

The distillate oil (35.38 kg) from the distillation after enzymatic treatment (column 5, Table 14) was then taken through a series of distillations in a short path distillation apparatus (temperature 120-141° C. and vacuum 0.01 mbar, VTA, model VK83-6-SKR-G with degasser). For each step a light fraction (20-30%) was removed as a distillate, while the residue was taken back to the next distillation steps. The composition of the residue (5.72 kg) after the last distillation step is shown in column 6 in Table 14.

An analysis of the distilled oils, both after ethylation and after enzyme treatment, surprisingly showed that the VLCPUFAs and VLCMUFAs can be distilled without being thermally degraded. It was also surprisingly found that the VLC fatty acids could be separated from glycerides and cholesterol ester by distillation providing an enriched composition of VLCUSFAs with a substantially reduced content of cholesterol.

### Cold Filtration:

3.5 kg of the residue oil from the above distillation (i.e. column 6, Table 14) was cooled down and stored overnight at 3° C., and then filtered. The fatty acid compositions (A %) of the starting oil (as of column 6, Table 14), the filtrate and the filter cake are shown in columns 2, 3 and 4 of Table 16, respectively.

TABLE 16

| Column 1 | 2 | 3 | 4 |
|---|---|---|---|
| | Start | Filtrate | Filter |
| | | 3° C. | cake |
| | | | 3° C. |
| Fatty acid | | | |
| C20:5 n3 | 0.24 | 0.29 | |
| C22:0 | 0.82 | 0.19 | 7.56 |
| C22:1 | 2.73 | 0.66 | |
| C22:5 n6 | 0.82 | 0.86 | 0.53 |
| C22:5n3 | 9.74 | 10.14 | 6.05 |
| C22:6n3 | 49.87 | 51.76 | 30.96 |
| C24:0 | 1.55 | 0 | 15.25 |
| C24:1 | 12.74 | 13.30 | 9.99 |
| C24:4n3 | 0.78 | 0.81 | 0.47 |
| C24:5n3 | 1.29 | 1.36 | 0.81 |
| C24:6n3 | 1.34 | 1.42 | |
| C26:1 | 0.95 | 1.00 | 0.67 |
| C26:3 | | | |
| C26:4n3 | 0.50 | 0.53 | 0.60 |
| C26:5n3 | 0.51 | 0.53 | 0.31 |
| C26:6n3 | 0.99 | 1.06 | 0.60 |
| C26:7n3 | 0.39 | 0.36 | 0.85 |
| C28:0 | | | |
| C28:1 | | | |
| C28:2 | | | |
| C28:4n3 | | | |
| C28:5n3 | 0.12 | 0.13 | 0.07 |
| C28:6n3 | 0.23 | 0.23 | 0.13 |
| C28:7n3 | 0.18 | 0.17 | 0.10 |
| C28:8n3 | 3.07 | 3.19 | 1.87 |
| VLC PUFA | 9.4 | 9.79 | 5.81 |
| VLC MUFA | 13.69 | 14.3 | 10.66 |

The above results show that saturated fatty acids can be fractioned from mono- and polyunsaturated fatty acids, also for very long chain fatty acids, by cold fractionation, as the saturated fatty acids tend to be removed in the filter cake.

### Bleaching

The oil (i.e. filtrate, Table 16, column 3) after the above cold filtration (3.03 kg) was bleached with 8% bleaching earth and 0.5% active carbon at 75° C. for 1 hour. The reaction mixture was cooled down and filtered. The oil after bleaching had a peroxide value of 0.2 meq/kg and an anisidine value of 14.7.

### Re-Esterification

2.75 kg of the above oil after bleaching was re-esterified to prepare triglyceride fatty acids, by mixing with 6.05% glycerol and 5% of Immobilised enzyme (Lipozyme 435, Novozymes) at 80° C. for 24 hours under vacuum (5-10 mbar). The reaction mixture was cooled down and filtered.

### Bleaching

The oil after the above re-esterification (2.31 kg) was bleached with 6% bleaching earth and stirred at 75° C. and 5-10 mbar for 1 hour. The reaction mixture was cooled down and filtered. The oil after bleaching (1.97 kg) had a peroxide value of 0.1 meq/kg and an anisidine value of 7.3.

### Distillation

The oil from the above bleaching (1.97 kg) was distilled using a short path distillation still (VTA, model VKL-70-4-SKR-T) at a temperature of 190° C., a flow of 3.5 ml/min and a pressure of 10⁻³ mbar. The residue (1.20 kg) was collected as the product, while the distillate (rich in ethyl ester and MG) was discarded. The ethyl ester and glyceride contents from before and after distillation (residue) are shown in Table Feil! Fant ikke referansekilden.17.

TABLE 17

| | Before Distillation | After |
|---|---|---|
| | (residue) | distillation |
| EE A % | 20.83 | 0 |
| MG A % | 8.12 | 0.4 |
| DG A % | 7.44 | 10.3 |
| TG A % | 63.05 | 89 |
| Oligomers A % | 0.6 | 0.6 |

### Deodorization

The above residue oil after distillation (1.20 kg), comprising fatty acids mainly on the triglyceride form, was deodorized with steam under vacuum (1-5 mbar) and 140° C. for 3 hours. The reaction mixture was cooled down and added mixed tocopherol. Analytical results of the deodorized “VLCFA triglyceride” oil (1.16 kg) are shown in Table Feil! Fant ikke referansekilden. 18 and Table 19 below.

TABLE 18

| Column 1 | 2 | 3 |
|---|---|---|
| Fatty acid | A % | Mg/g* |
| C20:5 n3 | 0.24 | 2 |
| C22:0 | 0.82 | |
| C22:1 | 3.01 | |
| C22:5 n6 | 0.82 | |
| C22:5 n3 | 9.88 | |
| C22:6 n3 | 49.53 | 452 |
| C24:0 | 0.11 | |
| C24:1 | 15.28 | 133 |
| C24:4n3 | 0.74 | |
| C24:5n3 | 1.31 | 12 |
| C24:6n3 | 1.41 | 13 |
| C26:1 | 1.29 | 11 |
| C26:3 n3 | | |
| C26:4n3 | 0.50 | 4 |
| C26:5n3 | 0.47 | 4 |
| C26:6n3 | 0.95 | 8 |
| C26:7n3 | 0.30 | 3 |
| C28:0 | | |
| C28:1 | | |
| C28:2 | | |
| C28:4n3 | | |
| C28:5n3 | 0.11 | 1 |
| C28:6n3 | 0.16 | 1 |
| C28:7n3 | 0.22 | 2 |
| C28:8n3 | 3.22 | 28 |
| VLC PUFA | 9.39 | 76 |
| VLC MUFA | 16.57 | 143 |

*mg/g analysis performed in similar way as: European Pharmacopoeia method 2.4.29 - “Composition of fatty acids in oils rich in Omega-3-acids.”, but with a modified temperature programme. Response factors for the VLC fatty acids were calculated by taking the response factor of DHA towards C23:0 and correct for theoretical response factors (based on number of active carbons and molecular weight) as described by Ackman (R.G. Ackman et al. The Journal of the American Oil Chemists' Society, vol 41, 1986, page 377-378).

TABLE 19

| Analysis | Result | | | |
|---|---|---|---|---|
| Appearance | Clear oil | USP | GOED | Ph.Eur. |
| Colour | 5 Gardner | | | |
| Total Cholesterol | 4.3 mg/g | | | |
| Peroxide value | 0.1 meq/g | <10 meq/g | <5 meq/g | <10.0 meq/g |
| Anisidine Value | 9 | <30 | <20 | <30.0 |
| Absorbance | 0.66 | <0.73 | | 0.73 |
| 233 nm | | | | |
| Triglycerides | 89 A % | | | |
| Diglycerides | 10.3 A % | | | |
| Monoglycerides | 0.4 A % | | | |
| Oligomers | 0.6 A % | <3.0 A % | | <3.0 A % |
| Mixed Tocopherol | 3.5 mg/g | | | |
| PCBs (6) | <0.002 mg/kg | | | |
| Dioxinlike PCBs | <0.21 pg/g | | <3 pg/g | |
| Dioxins + furans | <0.34 pg/g | <2.0 pg/g | <1.75 pg/g | |
| Dioxinlike | <0.55 pg/g | <10 pg/g | <3 pg/g | |
| PCBs + Dioxins/ | | | | |
| furans | | | | |
| Benzo(a)pyrene | <0.5 ng/g | | | |
| Sum PAH4 | <2.2 ng/g | | | |

The column called USP refers to the monograph of the US Pharmacopeia for omega-3 Acid Triglycerides and the column called GOED refers to the GOED voluntary monograph». The column called Ph. Eur. refers to the European Pharmacopoeia (Ph. Eur) (9th Edition 2019) monograph for Omega-3-acid triglycerides with regard to maximum limits for the tests. The VLCFA triglyceride product as described in Table 19 complies with the requirement of all three systems.

The skilled person would realise that protection against oxidation is less challenging for production scale volumes than for a low kg batch as exemplified in Table 19. Thus, it is likely that the results for the above type of tests would be even better, with lower values than those given in Table 19, when the process is carried out in commercial scale.

The above-prepared concentrate is highly purified and has been converted to a glyceride mixture. The purified VLCFA triglyceride product has a clear colour and has very low values for the oxidation parameters, cholesterol content and environmental pollutants.

The above examples and results show that enriched compositions comprising fatty acid mixtures of VLCMUFAs and VLCPUFAs as disclosed and claimed herein can be made.

1. A composition comprising a fatty acid mixture, wherein the fatty acid mixture comprises at least 4.0% by weight of very long chain monounsaturated fatty acids and at least 1.0% by weight of very long chain polyunsaturated fatty acids, wherein the fatty acids are derived from natural oils, and wherein the very long chain monounsaturated fatty acids and the very long chain polyunsaturated fatty acids have a chain length of 24 carbon atoms or more.

2. The composition of claim 1 wherein the natural oils are marine oils or oils from fresh water organisms.

3. The composition of claim 1 wherein the natural oil is selected from the group of fish oil, mollusc oil, crustacean oil, sea mammal oil, plankton oil, algal oil and microalgal oil.

4. The composition of claim 1 wherein the fatty acid mixture comprises at least 30% by weight of mono- and polyunsaturated fatty acids.

5. The composition of claim 1 wherein said fatty acid mixture comprises at least 8.0%, more preferably at least 15% by weight of very long chain monounsaturated fatty acids.

6. (canceled)

7. The composition of claim 1 wherein said fatty acid mixture comprises at least 1%, more preferably at least 6% by weight of very long chain monounsaturated fatty acids with chain length of more than 24 carbon atoms.

8. (canceled)

9. The composition of claim 1, wherein the fatty acid mixture comprises at least 2%, preferably at least 5%, and most preferably at least 10% by weight of one or more very long chain polyunsaturated fatty acids.

10. (canceled)

11. (canceled)

12. The composition of claim 1, wherein the fatty acid mixture comprises at least 10% by weight of one or more very long chain omega-3 polyunsaturated fatty acids.

13. The composition of claim 1 wherein the fatty acid mixture comprises very long chain monounsaturated fatty acids and very long chain polyunsaturated fatty acids in a total amount of at least 20%.

14. The composition of claim 1 wherein the fatty acid mixture comprises very long chain monounsaturated fatty acids and very long chain omega-3 polyunsaturated fatty acids in a total amount of at least 50%.

15. The composition of claim 1 wherein the fatty acid mixture comprises very long chain monounsaturated fatty acids and very long chain omega-3 polyunsaturated fatty acids in the weight ratio 3:1-1:2.

16. The composition of claim 1, wherein the fatty acid mixture comprises at least 5% by weight of one or more C28 very long chain polyunsaturated fatty acids.

17. The composition of claim 1, wherein the fatty acid mixture comprises at least 5% by weight of at least one of the very long chain fatty acids C28:6n3 and C28:8n3.

18. The composition of claim 1, wherein the fatty acid mixture comprises at least 5% by weight of the very long chain fatty acids C26:6n3.

19. The composition of claim 1, wherein the fatty acid mixture comprises at least 5% by weight of the very long chain fatty acids C24:5n3.

20. The composition of claim 1, wherein the fatty acid mixture further comprises at least 1% by weight of C18-C22 monounsaturated fatty acids.

21. The composition of claim 1, wherein the fatty acid mixture further comprises at least 1% by weight of C18-C22 polyunsaturated fatty acids.

22. The composition of claim 1 wherein said fatty acids are in the form of free fatty acids, fatty acid salts, mono-, di-, triglycerides, ethyl esters, wax esters, cholesteryl esters, ceramides, phospholipids or sphingomyelins, alone or in combination, and preferably are in the form of free fatty acids, fatty acid salts, ethyl esters, glycerides or wax esters.

23. (canceled)

24. The composition of claim 1, wherein the fatty acid mixture comprises less than 5 mg/g of cholesterol.

25. The composition of claim 1, wherein the fatty acid mixture has a colour Gardner below 8.

26. The composition of claim 1, wherein the fatty acid mixture comprises less than 2 μg/kg of benzo(A)pyrene (BAP) and/or below 10 μg/kg of polyaromatic hydrocarbons (4PAH).

27. The composition of claim 1 for use as a medicament, nutraceutical, food supplement, food additive or cosmetic product.

28. A method of making a composition comprising a fatty acid mixture comprising both very long chain polyunsaturated fatty acids (VLCPUFAs) and very long chain monounsaturated fatty acids (VLCMUFAs), wherein the fatty acid mixture is prepared from an oil material, comprising the steps of:
i) converting free cholesterol present in an oil material to cholesterol esters; and
ii) separating the cholesterol esters of step i) from very long chain fatty acid esters present in the material of step i).

29. The method of claim 28 wherein in step i) the oil material is brought in contact with an esterification catalyst, such as a lipase, converting the free cholesterol into cholesterol esters.

30. The method of claim 28 wherein in step ii) the oil material from step i) comprising cholesterol esters and fatty acid esters, is distilled.

31. The method of claim 28, wherein the fatty acid mixture comprises at least 4.0% by weight of very long chain monounsaturated fatty acids and at least 1.0% by weight of very long chain polyunsaturated fatty acids, wherein the fatty acids are derived from natural oils, wherein the very long chain monounsaturated fatty acids and the very long chain polyunsaturated fatty acids have a chain length of 24 carbon atoms or more, and wherein the composition comprises less than 5 mg/g of cholesterol.
